(19) United States
(12) Patent Application Publication
(10) Pub. No.: US 2023/0226180 A1
(43) Pub. Date: Jul. 20, 2023

(54) METHODS OF TREATING CANCER USING TIGIT INHIBITORS AND ANTI-CANCER AGENTS

(71) Applicant: Genentech, Inc., South San Francisco, CA (US)

(72) Inventor: Jane L. GROGAN, San Francisco, CA (US)

(21) Appl. No.: 18/154,581

(22) Filed: Jan. 13, 2023

Related U.S. Application Data
(63) Continuation of application No. 17/830,065, filed on Jun. 1, 2022, now abandoned.
Application No. 17/830,065 is a continuation of application No. 17/118,799, filed on Dec. 11, 2020, now abandoned.
Application No. 17/118,799 is a continuation of application No. 16/843,604, filed on Apr. 8, 2020, now abandoned.
Application No. 16/843,604 is a continuation of application No. 15/890,852, filed on Feb. 7, 2018, now abandoned.
Application No. 15/890,852 is a continuation of application No. 15/402,662, filed on Jan. 10, 2017, now abandoned.
Application No. 15/402,662 is a continuation of application No. PCT/US15/40770, filed on Jul. 16, 2015.
(60) Provisional application No. 62/025,394, filed on Jul. 16, 2014.

(51) Int. Cl. A61K 39/395; A61K 31/7088; A61K 45/06; C07K 16/28; A61K 38/19; A61K 38/20; A61K 38/21; A61K 47/68; A61P 35/00; A61K 39/00; A61K 38/00; C07K 16/30; C12N 15/113; C12N 15/115

(52) U.S. Cl. CPC A61K 39/39558; A61K 31/7088; A61K 45/06; C07K 16/2803; A61K 38/193; A61K 38/20; A61K 38/217; A61K 47/6803; A61K 47/6851; A61P 35/00; A61K 39/00117; A61K 39/001113; A61K 39/001124; A61K 39/001129; A61K 39/001106; A61K 39/001128; A61K 39/001138; A61K 39/001104; A61K 39/001119; A61K 39/00114; A61K 39/001141; A61K 39/001166; A61K 39/001109; A61K 38/00; A61K 39/0011; C07K 16/30; C12N 15/1138; C12N 15/115; Y02A 50/30; A61K 2039/505; A61K 2039/5158; A61K 2039/545; C07K 2317/51; C07K 2317/515; C07K 2317/56; C07K 2317/76; C12N 2310/11; C12N 2310/12; C12N 2310/14

(57) ABSTRACT

The present disclosure provides methods comprising administering to the individual an effective amount of an agent that decreases or inhibits TIGIT expression and/or activity and an anti-cancer agent and/or an anti-cancer therapy. Further provided are kits comprising an anti-cancer agent, an agent that decreases or inhibits TIGIT expression and/or activity, or both, as well as instructions for use thereof.

### CROSS-REFERENCE TO RELATED APPLICATIONS

This application is a continuation of U.S. application Ser. No. 17/830,065, filed Jun. 1, 2022, which is a continuation of U.S. application Ser. No. 17/118,799, filed Dec. 11, 2020, which is a continuation of U.S. application Ser. No. 16/843,604, filed Apr. 8, 2020, which is a continuation of U.S. application Ser. No. 15/890,852, filed Feb. 7, 2018, which is a continuation of U.S. application Ser. No. 15/402,662, filed Jan. 10, 2017, which is a continuation of International Patent Application No. PCT/US2015/040770, having an international filing date of Jul. 16, 2015, which claims the priority benefit of U.S. Provisional Application No. 62/025,394, filed Jul. 16, 2014, the contents of which are incorporated herein by reference in their entirety.

### SEQUENCE LISTING

The instant application contains a Sequence Listing which has been submitted electronically in XML format and is hereby incorporated by reference in its entirety. Said XML copy, created on Jan. 11, 2023, is named “50474-152007_Sequence_Listing_1_11_23” and is 24,626 bytes in size FIELD

The present disclosure relates to methods of treating cancers and/or tumor immunity by administering an agent that decreases or inhibits TIGIT expression and/or activity and an anti-cancer agent and/or an anti-cancer therapy.

### BACKGROUND

Many immune related diseases are known and have been extensively studied. Such diseases include immune-mediated inflammatory diseases, non-immune-mediated inflammatory diseases, infectious diseases, and immunodeficiency diseases. However, many cancers are also interconnected with the immune system, as various immune cells are able to mount an anti-cancer cell response, but cancer cells also possess mechanisms to suppress or evade these responses. Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, and also cancer cells. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response. Another subcategory of helper T cells are the follicular helper T cells (T_{Fh}) (for review, see Vineusa et al., Nat. Rev. Immunol. 5: 853-865 (2005)). Detectable by their characteristic expression of CXC-chemokine receptor 5 (Schaerli et al., J. Exp. Med. 192: 1553-62 (2000)), these cells have been found to produce IL-10 and possibly IL-21. T_{Fh} cells provide assistance to germinal-center B cells, particularly aiding the survival and propagation of B cells and potently inducing antibody production during coculture with B cells. They have also been implicated in tolerogenesis.

Regulatory T cells (T_{reg}) are a subset of helper T cells that play a critical role in inhibition of self-reactive immune responses and are often found in sites of chronic inflammation such as in tumor tissue (Wang, H. Y. & Wang, R. F., Curr Opin Immunol 19, 217-23 (2007)). T_{regs} perform their suppressive function on activated T cells through contact-dependent mechanisms and cytokine production (Fehervari, Z. & Sakaguchi, Curr Opin Immunol 16, 203-8 (2004)). T_{regs} also modulate immune responses by direct interaction with ligands on dendritic cells (DC). DCs are professional antigen-presenting cells capable of inducing immunity or tolerance against self or non-self antigens. DC-expanded T_{regs} suppress alloreactivity responses in vitro (Yamazaki, S. et al., Proc Nal Acad Sci USA 103, 2758-63 (2006); Ahn, J. S., Krishnadas, D. K. & Agrawal, Int Immunol 19, 227-37 (2007)), and when adoptively transferred, appropriate T_{regs} inhibited diabetes in NOD.scid mice (Tarbell, K. V. et al., J Exp Med 199, 1467-77 (2004)) or experimentally induced asthma (Lewkowich, I. P. et al. J Exp Med 202, 1549-61 (2005)).

In order to search for new co-stimulatory molecules expressed in T_{reg} cells searches were performed to identify genes specifically expressed in T cells (Abbas, A. R. et al., Genes Immun 6, 319-31 (2005)) that had both Ig domains and immunoreceptor tyrosine-based activation or inhibition (ITAM/ITIM) motifs. Through the intersection of these two genome-wide bioinformatics search strategies a novel cell surface-bound protein with the protein encoding an IgV domain, a transmembrane domain, and two putative immunoreceptor tyrosine inhibitory motifs was identified (see US patent publication no. US20040121370, incorporated herein by reference). The protein designated TIGIT (for T-Cell-Ig and ITIM domain) was shown to be expressed on T cells-particularly T_{reg} and memory cell subsets—as well as NK cells.

There is a need for new therapeutics and methods that modulate cells of the immune system to mount an anti-cancer response. In particular, agents that promote an anti-cancer function of NK cells, which can eradicate cancer cells, and/or memory T cells, which can maintain a lasting anti-cancer response, are highly advantageous.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

The present disclosure describes a combination treatment comprising an agent that decreases or inhibits TIGIT expression and/or activity and an anti-cancer agent and/or an anti-cancer therapy.

In certain aspects, the present disclosure provides a method for treating or delaying progression of cancer in an individual comprising administering to the individual an effective amount of an agent that decreases or inhibits TIGIT expression and/or activity and an anti-cancer agent or an anti-cancer therapy. In other aspects, the present disclosure provides use of an effective amount of an agent that decreases or inhibits TIGIT expression and/or activity in the manufacture of a medicament for treating or delaying progression of cancer in an individual, wherein the agent that decreases or inhibits TIGIT expression and/or activity is used in combination with an anti-cancer agent or an anti-cancer therapy. In other aspects, the present disclosure provides use of an effective amount of an anti-cancer agent in the manufacture of a medicament for treating or delaying progression of cancer in an individual, wherein the anti-cancer agent is used in combination with an agent that decreases or inhibits TIGIT expression and/or activity. In other aspects, the present disclosure provides a pharmaceutical composition comprising an agent that decreases or inhibits TIGIT expression and/or activity for use in treating or delaying progression of cancer in combination with an anti-cancer agent or an anti-cancer therapy. In other aspects, the present disclosure provides a pharmaceutical composition comprising an anti-cancer agent for use in treating or delaying progression of cancer in combination with an agent that decreases or inhibits TIGIT expression and/or activity.

In other aspects, the present disclosure provides a method for reducing or inhibiting cancer relapse or cancer progression in an individual comprising administering to the individual an effective amount of an agent that decreases or inhibits TIGIT expression and/or activity and an anti-cancer agent or an anti-cancer therapy. In other aspects, the present disclosure provides use of an effective amount of an agent that decreases or inhibits TIGIT expression and/or activity in the manufacture of a medicament for reducing or inhibiting cancer relapse or cancer progression in an individual, wherein the agent that decreases or inhibits TIGIT expression and/or activity is used in combination with an anti-cancer agent or an anti-cancer therapy. In other aspects, the present disclosure provides use of an effective amount of an anti-cancer agent in the manufacture of a medicament for reducing or inhibiting cancer relapse or cancer progression in an individual, wherein the anti-cancer agent is used in combination with an agent that decreases or inhibits TIGIT expression and/or activity. In other aspects, the present disclosure provides a pharmaceutical composition comprising an agent that decreases or inhibits TIGIT expression and/or activity for use in reducing or inhibiting cancer relapse or cancer progression in combination with an anti-cancer agent or an anti-cancer therapy. In other aspects, the present disclosure provides a pharmaceutical composition comprising an anti-cancer agent for use in reducing or inhibiting cancer relapse or cancer progression in combination with an agent that decreases or inhibits TIGIT expression and/or activity.

In other aspects, the present disclosure provides a method for treating or delaying progression of tumor immunity in an individual having cancer comprising administering to the individual an effective amount of an agent that decreases or inhibits TIGIT expression and/or activity and an anti-cancer agent or an anti-cancer therapy. In other aspects, the present disclosure provides use of an effective amount of an agent that decreases or inhibits TIGIT expression and/or activity in the manufacture of a medicament for treating or delaying progression of tumor immunity in an individual having cancer, wherein the agent that decreases or inhibits TIGIT expression and/or activity is used in combination with an anti-cancer agent or an anti-cancer therapy. In other aspects, the present disclosure provides use of an effective amount of an anti-cancer agent in the manufacture of a medicament for treating or delaying progression of tumor immunity in an individual having cancer, wherein the anti-cancer agent is used in combination with an agent that decreases or inhibits TIGIT expression and/or activity. In other aspects, the present disclosure provides a pharmaceutical composition comprising an agent that decreases or inhibits TIGIT expression and/or activity for use in treating or delaying progression of tumor immunity in combination with an anti-cancer agent or an anti-cancer therapy. In other aspects, the present disclosure provides a pharmaceutical composition comprising an anti-cancer agent for use in treating or delaying progression of tumor immunity in combination with an agent that decreases or inhibits TIGIT expression and/or activity.

In other aspects, the present disclosure provides a method of increasing, enhancing or stimulating an immune response or function in an individual having cancer comprising administering to the individual an effective amount of an agent that decreases or inhibits TIGIT expression and/or activity and an anti-cancer agent or an anti-cancer therapy. In other aspects, the present disclosure provides use of an effective amount of an agent that decreases or inhibits TIGIT expression and/or activity in the manufacture of a medicament for increasing, enhancing or stimulating an immune response or function in an individual having cancer, wherein the agent that decreases or inhibits TIGIT expression and/or activity is used in combination with an anti-cancer agent or an anti-cancer therapy. In other aspects, the present disclosure provides use of an effective amount of an anti-cancer agent in the manufacture of a medicament for increasing, enhancing or stimulating an immune response or function in an individual having cancer, wherein the anti-cancer agent is used in combination with an agent that decreases or inhibits TIGIT expression and/or activity. In other aspects, the present disclosure provides a pharmaceutical composition comprising an agent that decreases or inhibits TIGIT expression and/or activity for use in increasing, enhancing or stimulating an immune response or function in combination with an anti-cancer agent or an anti-cancer therapy. In other aspects, the present disclosure provides a pharmaceutical composition comprising an anti-cancer agent for use in increasing, enhancing or stimulating an immune response or function in combination with an agent that decreases or inhibits TIGIT expression and/or activity.

In other aspects, the present disclosure provides a combination comprising an effective amount of an agent that decreases or inhibits TIGIT expression and/or activity and an anti-cancer agent.

In certain embodiments that may be combined with any of the preceding embodiments, the individual has a T cell dysfunctional disorder. In certain embodiments that may be combined with any of the preceding embodiments, the T cell dysfunctional disorder is characterized by T cell anergy or decreased ability to secrete cytokines, proliferate or execute cytolytic activity. In certain embodiments that may be combined with any of the preceding embodiments, the T cell dysfunctional disorder is characterized by T cell exhaustion. In certain embodiments that may be combined with any of the preceding embodiments, the T cells are CD4+ and CD8+ T cells. In certain embodiments that may be combined with any of the preceding embodiments, the agent that decreases or inhibits TIGIT expression and/or activity is selected from the group consisting of an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof. In certain embodiments that may be combined with any of the preceding embodiments, the antagonist of TIGIT expression and/or activity is selected from the group consisting of a small molecule inhibitor, an inhibitory antibody or antigen-binding fragment thereof, an aptamer, an inhibitory nucleic acid, and an inhibitory polypeptide. In certain embodiments that may be combined with any of the preceding embodiments, the antagonist of PVR expression and/or activity is selected from the group consisting of a small molecule inhibitor, an inhibitory antibody or antigen-binding fragment thereof, an aptamer, an inhibitory nucleic acid, and an inhibitory polypeptide. In certain embodiments that may be combined with any of the preceding embodiments, the agent that inhibits and/or blocks the interaction of TIGIT with PVR is selected from the group consisting of a small molecule inhibitor, an inhibitory antibody or antigen-binding fragment thereof, an aptamer, an inhibitory nucleic acid, and an inhibitory polypeptide. In certain embodiments that may be combined with any of the preceding embodiments, the agent that inhibits and/or blocks the interaction of TIGIT with PVRL2 is selected from the group consisting of a small molecule inhibitor, an inhibitory antibody or antigen-binding fragment thereof, an aptamer, an inhibitory nucleic acid, and an inhibitory polypeptide. In certain embodiments that may be combined with any of the preceding embodiments, the agent that inhibits and/or blocks the interaction of TIGIT with PVRL3 is selected from the group consisting of a small molecule inhibitor, an inhibitory antibody or antigen-binding fragment thereof, an aptamer, an inhibitory nucleic acid, and an inhibitory polypeptide. In certain embodiments that may be combined with any of the preceding embodiments, the agent that inhibits and/or blocks the intracellular signaling mediated by TIGIT binding to PVR is selected from the group consisting of a small molecule inhibitor, an inhibitory antibody or antigen-binding fragment thereof, an aptamer, an inhibitory nucleic acid, and an inhibitory polypeptide. In certain embodiments that may be combined with any of the preceding embodiments, the agent that inhibits and/or blocks the intracellular signaling mediated by TIGIT binding to PVRL2 is selected from the group consisting of a small molecule inhibitor, an inhibitory antibody or antigen-binding fragment thereof, an aptamer, an inhibitory nucleic acid, and an inhibitory polypeptide. In certain embodiments that may be combined with any of the preceding embodiments, the agent that inhibits and/or blocks the intracellular signaling mediated by TIGIT binding to PVRL3 is selected from the group consisting of a small molecule inhibitor, an inhibitory antibody or antigen-binding fragment thereof, an aptamer, an inhibitory nucleic acid, and an inhibitory polypeptide. In certain embodiments that may be combined with any of the preceding embodiments, the inhibitory nucleic acid is selected from the group consisting of an antisense polynucleotide, an interfering RNA, a catalytic RNA, and an RNA-DNA chimera. In certain embodiments that may be combined with any of the preceding embodiments, the inhibitory antibody or antigen-binding fragment thereof is an anti-TIGIT antibody or antigen-binding fragment thereof. In certain embodiments that may be combined with any of the preceding embodiments, the anti-TIGIT antibody or antigen-binding fragment thereof is selected from the group consisting of a humanized antibody, a chimeric antibody, a bispecific antibody, a heteroconjugate antibody, and an immunotoxin. In certain embodiments that may be combined with any of the preceding embodiments, the anti-TIGIT antibody or antigen-binding fragment thereof comprises at least one HVR comprising an amino acid sequence selected from the amino acid sequences (1) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (2) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12). In certain embodiments that may be combined with any of the preceding embodiments, the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGT KLEIKR (SEQ ID NO:13) or DVVLTQTPLSLSVSFGDQVSISCRSSQSLVNSYGNTFLSWYLHKPGQSPQLLIFGISNRF SGVPDRFSGSGSGTDFTLKISTIKPEDLGMYYCLQGTHQPPTFGPGTKLEVK (SEQ ID NO:14). In certain embodiments that may be combined with any of the preceding embodiments, the anti-TIGIT antibody or antigen-binding fragment thereof, wherein the antibody heavy chain comprises the amino acid sequence set forth in EVQLVESGGGLTQPGKSLKLSCEASGFTFSSFTMHWVRQSPGKGLEWVAFIRSGSGIV FYADAVRGRFTISRDNAKNLLFLQMNDLKSEDTAMYYCARRPLGHNTFDSWGQGTL VTVSS (SEQ ID NO:15) or EVQLQQSGPELVKPGTSMKISCKASGYSFTGHLMNWVKQSHGKNLEWIGLIIPYNGGT SYNQKFKGKATLTVDKSSSTAYMELLSLTSDDSAVYFCSRGLRGFYAMDYWGQGTS VTVSS (SEQ ID NO:16). In certain embodiments that may be combined with any of the preceding embodiments, the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGT KLEIKR (SEQ ID NO:13) or DVVLTQTPLSLSVSFGDQVSISCRSSQSLVNSYGNTFLSWYLHKPGQSPQLLIFGISNRF SGVPDRFSGSGSGTDFTLKISTIKPEDLGMYYCLQGTHQPPTFGPGTKLEVK (SEQ ID NO:14), and the antibody heavy chain comprises the amino acid sequence set forth in EVQLVESGGGLTQPGKSLKLSCEASGFTFSSFTMHWVRQSPGKGLEWVAFIRSGSGIV FYADAVRGRFTISRDNAKNLLFLQMNDLKSEDTAMYYCARRPLGHNTFDSWGQGTL VTVSS (SEQ ID NO:15) or EVQLQQSGPELVKPGTSMKISCKASGYSFTGHLMNWVKQSHGKNLEWIGLIIPYNGGT SYNQKFKGKATLTVDKSSSTAYMELLSLTSDDSAVYFCSRGLRGFYAMDYWGQGTS VTVSS (SEQ ID NO: 16). In certain embodiments that may be combined with any of the preceding embodiments, the anti-TIGIT antibody or antigen-binding fragment thereof comprises at least one HVR that is at least 90% identical to an HVR set forth in any one of (1) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (2) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12). In certain embodiments that may be combined with any of the preceding embodiments, the anti-TIGIT antibody or fragment thereof comprises the light chain comprising amino acid sequences at least 90% identical to the amino acid sequences set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGT KLEIKR (SEQ ID NO:13) or DVVLTQTPLSLSVSFGDQVSISCRSSQSLVNSYGNTFLSWYLHKPGQSPQLLIFGISNRF SGVPDRFSGSGSGTDFTLKISTIKPEDLGMYYCLQGTHQPPTFGPGTKLEVK (SEQ ID NO:14); and/or the heavy chain comprising amino acid sequences at least 90% identical to the amino acid sequences set forth in EVQLVESGGGLTQPGKSLKLSCEASGFTFSSFTMHWVRQSPGKGLEWVAFIRSGSGIV FYADAVRGRFTISRDNAKNLLFLQMNDLKSEDTAMYYCARRPLGHNTFDSWGQGTL VTVSS (SEQ ID NO:15) or EVQLQQSGPELVKPGTSMKISCKASGYSFTGHLMNWVKQSHGKNLEWIGLIIPYNGGT SYNQKFKGKATLTVDKSSSTAYMELLSLTSDDSAVYFCSRGLRGFYAMDYWGQGTS VTVSS (SEQ ID NO:16). In certain embodiments that may be combined with any of the preceding embodiments, the method comprises administering to the individual an effective amount of an agent that decreases or inhibits TIGIT expression and/or activity, an anti-cancer agent, and an anti-cancer therapy. In certain embodiments that may be combined with any of the preceding embodiments, the anti-cancer agent is one or more anti-cancer agents. In certain embodiments that may be combined with any of the preceding embodiments, the one or more anti-cancer agents are two or more, three or more, four or more, five or more, six or more, seven or more, eight or more, nine or more, or ten or more anti-cancer agents. In certain embodiments that may be combined with any of the preceding embodiments, the one or more anti-cancer agents are two or more anti-cancer agents. In certain embodiments that may be combined with any of the preceding embodiments, the one or more anti-cancer agents are three or more anti-cancer agents. In certain embodiments that may be combined with any of the preceding embodiments, the one or more anti-cancer agents are four or more anti-cancer agents. In certain embodiments that may be combined with any of the preceding embodiments, the one or more anti-cancer agents are five or more anti-cancer agents. In certain embodiments that may be combined with any of the preceding embodiments, the anti-cancer therapy is one or more anti-cancer therapies. In certain embodiments that may be combined with any of the preceding embodiments, the one or more anti-cancer therapies are two or more, three or more, four or more, five or more, six or more, seven or more, eight or more, nine or more, or ten or more anti-cancer therapies. In certain embodiments that may be combined with any of the preceding embodiments, the one or more anti-cancer therapies are two or more anti-cancer therapies. In certain embodiments that may be combined with any of the preceding embodiments, the one or more anti-cancer therapies are three or more anti-cancer therapies. In certain embodiments that may be combined with any of the preceding. embodiments, the one or more anti-cancer therapies are four or more anti-cancer therapies. In certain embodiments that may be combined with any of the preceding. embodiments, the one or more anti-cancer therapies are five or more anti-cancer therapies. In certain embodiments that may be combined with any of the preceding embodiments, the anti-cancer therapy is selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, and combinations thereof. In certain embodiments that may be combined with any of the preceding embodiments, the one or more anti-cancer therapies are selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, and combinations thereof. In certain embodiments that may be combined with any of the preceding embodiments, the anti-cancer agent is selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof. In certain embodiments that may be combined with any of the preceding embodiments, the one or more anti-cancer agents are selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof. In certain embodiments that may be combined with any of the preceding embodiments, the method comprises administering to the individual an effective amount of an agent that decreases or inhibits TIGIT expression and/or activity, an anti-cancer agent, and an anti-cancer therapy. In certain embodiments that may be combined with any of the preceding embodiments, the chemotherapeutic or growth inhibitory agent is selected from the group consisting of an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof. In certain embodiments that may be combined with any of the preceding embodiments, the targeted therapeutic agent is selected from the group consisting of a B-raf inhibitor, a MEK inhibitor, a K-ras inhibitor, a c-Met inhibitor, an Alk inhibitor, a phosphatidylinositol 3-kinase inhibitor, an Akt inhibitor, an mTOR inhibitor, a dual phosphatidylinositol 3-kinase/mTOR inhibitor, and combinations thereof. In certain embodiments that may be combined with any of the preceding embodiments, the T cell expressing a chimeric antigen receptor comprises a dominant-negative TGF beta receptor. In certain embodiments that may be combined with any of the preceding embodiments, the antibody or antigen-binding fragment thereof is selected from the group consisting of alemtuzumab, bevacizumab, cetuximab, panitumumab, rituximab, pertuzumab, trastuzumab, tositumomab, apolizumab, aselizumab, atlizumab, bapineuzumab, cedelizumab, certolizumab pegol, cidfusituzumab, cidtuzumab, clivatuzumab, daclizumab, eculizumab, efalizumab, epratuzumab, erlizumab, felvizumab, fontolizumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pertuzumab, pexelizumab, ralivizumab, ranibizumab, reslivizumab, reslizumab, resyvizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, anti-IL-12, and anti-IL-17. In certain embodiments that may be combined with any of the preceding embodiments, the antibody or antigen-binding fragment thereof specifically binds to a target selected from the group consisting of CD52, VEGF-A, EGFR, CD20, HER2, HLA-DRB, CD62L, IL-6R, amyloid beta, CD44, CanAg, CD4, TNF alpha, IL-2, CD25, complement C5, CD11a, CD22, CD18, respiratory syncytial virus F, interferon gamma, CD33, CEACAM5, IL-5, integrin alpha 4, IgE, IL-4, IL-5, CD154, FAP, CD2, MUC-1, AFP, integrin aIlbP3, ClfA, IL6R, CD40L, EpCAM, Shiga-like toxin II, IL-12, IL-23, IL-17, and CD3. In certain embodiments that may be combined with any of the preceding embodiments, the antibody-drug conjugate comprises a drug selected from the group consisting of mertansine, monomethyl auristatin E, a calicheamicin, an esperamicin, and a radioisotope chelator. In certain embodiments that may be combined with any of the preceding embodiments, the angiogenesis inhibitor is selected from the group consisting of a VEGF antagonist and an angiopoietin 2 antagonist. In certain embodiments that may be combined with any of the preceding embodiments, the antineoplastic agent is selected from the group consisting of an agent targeting CSF-1R, an interferon, GM-CSF, IL-2, IL-12, and an antibody targeting CD20. In certain embodiments that may be combined with any of the preceding embodiments, the cancer vaccine is selected from the group consisting of a peptide cancer vaccine, a personalized peptide vaccine, a multivalent long peptide vaccine, a multi-peptide vaccine, a peptide cocktail vaccine, a hybrid peptide vaccine, and a peptide-pulsed dendritic cell vaccine. In certain embodiments that may be combined with any of the preceding embodiments, the anti-cancer agent is selected from the group consisting of a TLR agonist, tumor necrosis factor alpha, IL-1, HMGB1, an IL-10 antagonist, an IL-4 antagonist, an IL-13 antagonist, a treatment targeting CX3CL1, a treatment targeting CXCL9, a treatment targeting CXCL10, a treatment targeting CCL5, an LFA-1 agonist, an ICAM1 agonist, and a Selectin agonist. In certain embodiments that may be combined with any of the preceding embodiments, the one or more anti-cancer agents are selected from the group consisting of a TLR agonist, tumor necrosis factor alpha, IL-1, HMGB1, an IL-10 antagonist, an IL-4 antagonist, an IL-13 antagonist, a treatment targeting CX3CL1, a treatment targeting CXCL9, a treatment targeting CXCL10, a treatment targeting CCL5, an LFA-1 agonist, an ICAM1 agonist, a Selectin agonist, and combinations thereof. In certain embodiments that may be combined with any of the preceding embodiments, the agent that decreases or inhibits TIGIT expression and/or activity is administered continuously. In certain embodiments that may be combined with any of the preceding embodiments, the agent that decreases or inhibits TIGIT expression and/or activity is administered intermittently. In certain embodiments that may be combined with any of the preceding embodiments, the anti-cancer agent or anti-cancer therapy is administered continuously. In certain embodiments that may be combined with any of the preceding embodiments, the anti-cancer agent or anti-cancer therapy is administered intermittently. In certain embodiments that may be combined with any of the preceding embodiments, the agent that decreases or inhibits TIGIT expression and/or activity is administered before the anti-cancer agent or anti-cancer therapy. In certain embodiments that may be combined with any of the preceding embodiments, the agent that decreases or inhibits TIGIT expression and/or activity is administered simultaneous with the anti-cancer agent or anti-cancer therapy. In certain embodiments that may be combined with any of the preceding embodiments, the agent that decreases or inhibits TIGIT expression and/or activity is administered after the anti-cancer agent or anti-cancer therapy. In certain embodiments that may be combined with any of the preceding embodiments, the cancer is selected from the group consisting of non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycosis fungoides, merkel cell cancer, and other hematologic malignancies. In certain embodiments that may be combined with any of the preceding embodiments, the cancer has elevated levels of T cell infiltration. In certain embodiments that may be combined with any of the preceding embodiments, activated CD4 and/or CD8 T cells in the individual are characterized by γ-IFN⁺ producing CD4 and/or CD8 T cells and/or enhanced cytolytic activity relative to prior to the administration of the combination. In certain embodiments that may be combined with any of the preceding embodiments, the CD4 and/or CD8 T cells exhibit increased release of cytokines selected from the group consisting of IFN-γ, TNF-α and interleukins. In certain embodiments that may be combined with any of the preceding embodiments, the CD4 and/or CD8 T cells are effector memory T cells. In certain embodiments that may be combined with any of the preceding embodiments, the CD4 and/or CD8 effector memory T cells are characterized by having the expression of CD₄₄^{high} CD62L^{low}.

In other aspects, the present disclosure provides a kit comprising an anti-cancer agent and a package insert comprising instructions for using the anti-cancer agent in combination with an agent that decreases or inhibits TIGIT expression and/or activity to treat or delay progression of cancer in an individual. In other aspects, the present disclosure provides a kit comprising an anti-cancer agent and an agent that decreases or inhibits TIGIT expression and/or activity, and a package insert comprising instructions for using the anti-cancer agent and the agent that decreases or inhibits TIGIT expression and/or activity to treat or delay progression of cancer in an individual. In other aspects, the present disclosure provides a kit comprising an agent that decreases or inhibits TIGIT expression and/or activity and a package insert comprising instructions for using the agent that decreases or inhibits TIGIT expression and/or activity in combination with an anti-cancer agent or an anti-cancer therapy to treat or delay progression of cancer in an individual.

In other aspects, the present disclosure provides a kit comprising an anti-cancer agent and a package insert comprising instructions for using the anti-cancer agent in combination with an agent that decreases or inhibits TIGIT expression and/or activity to reduce or inhibit cancer relapse or cancer progression in an individual having cancer. In other aspects, the present disclosure provides a kit comprising an anti-cancer agent and an agent that decreases or inhibits TIGIT expression and/or activity, and a package insert comprising instructions for using the anti-cancer agent and the agent that decreases or inhibits TIGIT expression and/or activity to reduce or inhibit cancer relapse or cancer progression in an individual having cancer. In other aspects, the present disclosure provides a kit comprising an agent that decreases or inhibits TIGIT expression and/or activity and a package insert comprising instructions for using the agent that decreases or inhibits TIGIT expression and/or activity in combination with an anti-cancer agent or an anti-cancer therapy to reduce or inhibit cancer relapse or cancer progression in an individual having cancer.

In other aspects, the present disclosure provides a kit comprising an anti-cancer agent and a package insert comprising instructions for using the anti-cancer agent in combination with an agent that decreases or inhibits TIGIT expression and/or activity to treat or delay progression of tumor immunity in an individual having cancer. In other aspects, the present disclosure provides a kit comprising an anti-cancer agent and an agent that decreases or inhibits TIGIT expression and/or activity, and a package insert comprising instructions for using the anti-cancer agent and the agent that decreases or inhibits TIGIT expression and/or activity to treat or delay progression of tumor immunity in an individual having cancer. In other aspects, the present disclosure provides a kit comprising an agent that decreases or inhibits TIGIT expression and/or activity and a package insert comprising instructions for using the agent that decreases or inhibits TIGIT expression and/or activity in combination with an anti-cancer agent or an anti-cancer therapy to treat or delay progression of tumor immunity in an individual having cancer.

In other aspects, the present disclosure provides a kit comprising an anti-cancer agent and a package insert comprising instructions for using the anti-cancer agent in combination with an agent that decreases or inhibits TIGIT expression and/or activity to increase, enhance, or stimulate an immune response or function in an individual having cancer. In other aspects, the present disclosure provides a kit comprising an anti-cancer agent and an agent that decreases or inhibits TIGIT expression and/or activity, and a package insert comprising instructions for using the anti-cancer agent and the agent that decreases or inhibits TIGIT expression and/or activity to increase, enhance, or stimulate an immune response or function in an individual having cancer. In other aspects, the present disclosure provides a kit comprising an agent that decreases or inhibits TIGIT expression and/or activity and a package insert comprising instructions for using the agent that decreases or inhibits TIGIT expression and/or activity in combination with an anti-cancer agent or an anti-cancer therapy to increase, enhance, or stimulate an immune response or function in an individual having cancer.

In certain embodiments that may be combined with any of the preceding embodiments, the one or more anti-cancer agents are two or more, three or more, four or more, five or more, six or more, seven or more, eight or more, nine or more, or ten or more anti-cancer agents. In certain embodiments that may be combined with any of the preceding embodiments, the one or more anti-cancer agents are two or more anti-cancer agents. In certain embodiments that may be combined with any of the preceding embodiments, the one or more anti-cancer agents are three or more anti-cancer agents. In certain embodiments that may be combined with any of the preceding embodiments, the one or more anti-cancer agents are four or more anti-cancer agents. In certain embodiments that may be combined with any of the preceding embodiments, the one or more anti-cancer agents are five or more anti-cancer agents. In certain embodiments that may be combined with any of the preceding embodiments, the anti-cancer therapy is one or more anti-cancer therapies. In certain embodiments that may be combined with any of the preceding embodiments, the one or more anti-cancer therapies are two or more, three or more, four or more, five or more, six or more, seven or more, eight or more, nine or more, or ten or more anti-cancer therapies. In certain embodiments that may be combined with any of the preceding embodiments, the one or more anti-cancer therapies are two or more anti-cancer therapies. In certain embodiments that may be combined with any of the preceding. embodiments, the one or more anti-cancer therapies are three or more anti-cancer therapies. In certain embodiments that may be combined with any of the preceding embodiments, the one or more anti-cancer therapies are four or more anti-cancer therapies. In certain embodiments that may be combined with any of the preceding embodiments, the one or more anti-cancer therapies are five or more anti-cancer therapies. In certain embodiments that may be combined with any of the preceding embodiments, the anti-cancer therapy is selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, and combinations thereof. In certain embodiments that may be combined with any of the preceding embodiments, the one or more anti-cancer therapies are selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, and combinations thereof. In certain embodiments that may be combined with any of the preceding embodiments, the agent that decreases or inhibits TIGIT expression and/or activity is selected from the group consisting of an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, and an agent that inhibits and/or blocks the intracellular signaling mediated by TIGIT binding to PVRL3. In certain embodiments that may be combined with any of the preceding embodiments, the antagonist of TIGIT expression and/or activity is an anti-TIGIT antibody or antigen-binding fragment thereof. In certain embodiments that may be combined with any of the preceding embodiments, the anti-TIGIT antibody or antigen-binding fragment thereof comprises at least one HVR comprising an amino acid sequence selected from the amino acid sequences (1) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (2) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12). In certain embodiments that may be combined with any of the preceding embodiments, the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGT KLEIKR (SEQ ID NO:13) or DVVLTQTPLSLSVSFGDQVSISCRSSQSLVNSYGNTFLSWYLHKPGQSPQLLIFGISNRF SGVPDRFSGSGSGTDFTLKISTIKPEDLGMYYCLQGTHQPPTFGPGTKLEVK (SEQ ID NO:14). In certain embodiments that may be combined with any of the preceding embodiments, the anti-TIGIT antibody or antigen-binding fragment thereof, wherein the antibody heavy chain comprises the amino acid sequence set forth in EVQLVESGGGLTQPGKSLKLSCEASGFTFSSFTMHWVRQSPGKGLEWVAFIRSGSGIV FYADAVRGRFTISRDNAKNLLFLQMNDLKSEDTAMYYCARRPLGHNTFDSWGQGTL VTVSS (SEQ ID NO:15) or EVQLQQSGPELVKPGTSMKISCKASGYSFTGHLMNWVKQSHGKNLEWIGLIIPYNGGT SYNQKFKGKATLTVDKSSSTAYMELLSLTSDDSAVYFCSRGLRGFYAMDYWGQGTS VTVSS (SEQ ID NO:16). In certain embodiments that may be combined with any of the preceding embodiments, the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGT KLEIKR (SEQ ID NO:13) or DVVLTQTPLSLSVSFGDQVSISCRSSQSLVNSYGNTFLSWYLHKPGQSPQLLIFGISNRF SGVPDRFSGSGSGTDFTLKISTIKPEDLGMYYCLQGTHQPPTFGPGTKLEVK (SEQ ID NO:14), and the antibody heavy chain comprises the amino acid sequence set forth in EVQLVESGGGLTQPGKSLKLSCEASGFTFSSFTMHWVRQSPGKGLEWVAFIRSGSGIV FYADAVRGRFTISRDNAKNLLFLQMNDLKSEDTAMYYCARRPLGHNTFDSWGQGTL VTVSS (SEQ ID NO:15) or EVQLQQSGPELVKPGTSMKISCKASGYSFTGHLMNWVKQSHGKNLEWIGLIIPYNGGT SYNQKFKGKATLTVDKSSSTAYMELLSLTSDDSAVYFCSRGLRGFYAMDYWGQGTS VTVSS (SEQ ID NO: 16). In certain embodiments that may be combined with any of the preceding embodiments, the anti-TIGIT antibody or antigen-binding fragment thereof comprises at least one HVR that is at least 90% identical to an HVR set forth in any one of (1) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (2) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12). In certain embodiments that may be combined with any of the preceding embodiments, the anti-TIGIT antibody or fragment thereof comprises the light chain comprising amino acid sequences at least 90% identical to the amino acid sequences set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGT KLEIKR (SEQ ID NO:13) or DVVLTQTPLSLSVSFGDQVSISCRSSQSLVNSYGNTFLSWYLHKPGQSPQLLIFGISNRF SGVPDRFSGSGSGTDFTLKISTIKPEDLGMYYCLQGTHQPPTFGPGTKLEVK (SEQ ID NO:14); and/or the heavy chain comprising amino acid sequences at least 90% identical to the amino acid sequences set forth in EVQLVESGGGLTQPGKSLKLSCEASGFTFSSFTMHWVRQSPGKGLEWVAFIRSGSGIV FYADAVRGRFTISRDNAKNLLFLQMNDLKSEDTAMYYCARRPLGHNTFDSWGQGTL VTVSS (SEQ ID NO:15) or EVQLQQSGPELVKPGTSMKISCKASGYSFTGHLMNWVKQSHGKNLEWIGLIIPYNGGT SYNQKFKGKATLTVDKSSSTAYMELLSLTSDDSAVYFCSRGLRGFYAMDYWGQGTS VTVSS (SEQ ID NO:16). In certain embodiments that may be combined with any of the preceding embodiments, the anti-cancer agent is selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof. In certain embodiments that may be combined with any of the preceding embodiments, the one or more anti-cancer agents are selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof. In certain embodiments that may be combined with any of the preceding embodiments, the chemotherapeutic or growth inhibitory agent is selected from the group consisting of an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof. In certain embodiments that may be combined with any of the preceding embodiments, the targeted therapeutic agent is selected from the group consisting of a B-raf inhibitor, a MEK inhibitor, a K-ras inhibitor, a c-Met inhibitor, an Alk inhibitor, a phosphatidylinositol 3-kinase inhibitor, an Akt inhibitor, an mTOR inhibitor, a dual phosphatidylinositol 3-kinase/mTOR inhibitor, and combinations thereof. In certain embodiments that may be combined with any of the preceding embodiments, the T cell expressing a chimeric antigen receptor comprises a dominant-negative TGF beta receptor. In certain embodiments that may be combined with any of the preceding embodiments, the antibody or antigen-binding fragment thereof is selected from the group consisting of alemtuzumab, bevacizumab, cetuximab, panitumumab, rituximab, pertuzumab, trastuzumab, tositumomab, apolizumab, aselizumab, atlizumab, bapineuzumab, cedelizumab, certolizumab pegol, cidfusituzumab, cidtuzumab, clivatuzumab, daclizumab, eculizumab, efalizumab, epratuzumab, erlizumab, felvizumab, fontolizumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pertuzumab, pexelizumab, ralivizumab, ranibizumab, reslivizumab, reslizumab, resyvizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, anti-IL-12, and anti-IL-17. In certain embodiments that may be combined with any of the preceding embodiments, the antibody or antigen-binding fragment thereof specifically binds to a target selected from the group consisting of CD52, VEGF-A, EGFR, CD20, HER2, HLA-DRB, CD62L, IL-6R, amyloid beta, CD44, CanAg, CD4, TNF alpha, IL-2, CD25, complement C5, CD11a, CD22, CD18, respiratory syncytial virus F, interferon gamma, CD33, CEACAM5, IL-5, integrin alpha 4, IgE, IL-4, IL-5, CD154, FAP, CD2, MUC-1, AFP, integrin αIIbβ3, ClfA, IL6R, CD40L, EpCAM, Shiga-like toxin II, IL-12, IL-23, IL-17, and CD3. In certain embodiments that may be combined with any of the preceding embodiments, the antibody-drug conjugate comprises a drug selected from the group consisting of mertansine, monomethyl auristatin E, calicheamicin, esperamicin, and a radioisotope chelator. In certain embodiments that may be combined with any of the preceding embodiments, the angiogenesis inhibitor is selected from the group consisting of a VEGF antagonist and an angiopoietin 2 antagonist. In certain embodiments that may be combined with any of the preceding embodiments, the antineoplastic agent is selected from the group consisting of an agent targeting CSF-1R, an interferon, GM-CSF, IL-2, IL-12, and an antibody targeting CD20. In certain embodiments that may be combined with any of the preceding embodiments, the cancer vaccine is selected from the group consisting of a peptide cancer vaccine, a personalized peptide vaccine, a multivalent long peptide vaccine, a multi-peptide vaccine, a peptide cocktail vaccine, a hybrid peptide vaccine, and a peptide-pulsed dendritic cell vaccine. In certain embodiments that may be combined with any of the preceding embodiments, the anti-cancer agent is selected from the group consisting of a TLR agonist, tumor necrosis factor alpha, IL-1, HMGB1, an IL-10 antagonist, an IL-4 antagonist, an IL-13 antagonist, a treatment targeting CX3CL1, a treatment targeting CXCL9, a treatment targeting CXCL10, a treatment targeting CCL5, an LFA-1 agonist, an ICAM1 agonist, and a Selectin agonist. In certain embodiments that may be combined with any of the preceding embodiments, the one or more anti-cancer agents are selected from the group consisting of a TLR agonist, tumor necrosis factor alpha, IL-1, HMGB1, an IL-10 antagonist, an IL-4 antagonist, an IL-13 antagonist, a treatment targeting CX3CL1, a treatment targeting CXCL9, a treatment targeting CXCL10, a treatment targeting CCL5, an LFA-1 agonist, an ICAM1 agonist, a Selectin agonist, and combinations thereof.

### DETAILED DESCRIPTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

The term “antagonist” is used in the broadest sense, and includes any molecule that partially or fully blocks, inhibits, or neutralizes a biological activity of a native polypeptide disclosed herein. In a similar manner, the term “agonist” is used in the broadest sense and includes any molecule that mimics a biological activity of a native polypeptide disclosed herein. Suitable agonist or antagonist molecules specifically include agonist or antagonist antibodies or antibody fragments, fragments or amino acid sequence variants of native polypeptides, peptides, antisense oligonucleotides, small organic molecules, etc. Methods for identifying agonists or antagonists of a polypeptide may comprise contacting a polypeptide with a candidate agonist or antagonist molecule and measuring a detectable change in one or more biological activities normally associated with the polypeptide.

The term “aptamer” refers to a nucleic acid molecule that is capable of binding to a target molecule, such as a polypeptide. For example, an aptamer of the invention can specifically bind to a TIGIT polypeptide, or to a molecule in a signaling pathway that modulates the expression of TIGIT. The generation and therapeutic use of aptamers are well established in the art. See, e.g., U.S. Pat. No. 5,475,096, and the therapeutic efficacy of Macugen® (Eyetech, New York) for treating age-related macular degeneration.

The terms “TIGIT antagonist” and “antagonist of TIGIT activity or TIGIT expression” are used interchangeably and refer to a compound that interferes with the normal functioning of TIGIT, either by decreasing transcription or translation of TIGIT-encoding nucleic acid, or by inhibiting or blocking TIGIT polypeptide activity, or both. Examples of TIGIT antagonists include, but are not limited to, antisense polynucleotides, interfering RNAs, catalytic RNAs, RNA-DNA chimeras, TIGIT-specific aptamers, anti-TIGIT antibodies, TIGIT-binding fragments of anti-TIGIT antibodies, TIGIT-binding small molecules, TIGIT-binding peptides, and other polypeptides that specifically bind TIGIT (including, but not limited to, TIGIT-binding fragments of one or more TIGIT ligands, optionally fused to one or more additional domains), such that the interaction between the TIGIT antagonist and TIGIT results in a reduction or cessation of TIGIT activity or expression. It will be understood by one of ordinary skill in the art that in some instances, a TIGIT antagonist may antagonize one TIGIT activity without affecting another TIGIT activity. For example, a desirable TIGIT antagonist for use in certain of the methods herein is a TIGIT antagonist that antagonizes TIGIT activity in response to one of PVR interaction, PVRL3 interaction, or PVRL2 interaction, e.g., without affecting or minimally affecting any of the other TIGIT interactions.

The terms “PVR antagonist” and “antagonist of PVR activity or PVR expression” are used interchangeably and refer to a compound that interferes with the normal functioning of PVR, either by decreasing transcription or translation of PVR-encoding nucleic acid, or by inhibiting or blocking PVR polypeptide activity, or both. Examples of PVR antagonists include, but are not limited to, antisense polynucleotides, interfering RNAs, catalytic RNAs, RNA-DNA chimeras, PVR-specific aptamers, anti-PVR antibodies, PVR-binding fragments of anti-PVR antibodies, PVR-binding small molecules, PVR-binding peptides, and other polypeptides that specifically bind PVR (including, but not limited to, PVR-binding fragments of one or more PVR ligands, optionally fused to one or more additional domains), such that the interaction between the PVR antagonist and PVR results in a reduction or cessation of PVR activity or expression. It will be understood by one of ordinary skill in the art that in some instances, a PVR antagonist may antagonize one PVR activity without affecting another PVR activity, e.g., a PVR antagonist that antagonizes an interaction between PVR and TIGIT without antagonizing an interaction between PVR and a molecule other than TIGIT, or a PVR antagonist that antagonizes PVR activity in response to TIGIT interaction without antagonizing PVR activity in response to interaction with a molecule other than TIGIT.

The term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth.

The term “dysfunctional”, as used herein, also includes refractory or unresponsive to antigen recognition, specifically, impaired capacity to translate antigen recognition into down-stream T-cell effector functions, such as proliferation, cytokine production (e.g., IL-2) and/or target cell killing.

The term “anergy” refers to the state of unresponsiveness to antigen stimulation resulting from incomplete or insufficient signals delivered through the T-cell receptor (e.g. increase in intracellular Ca⁺² in the absence of ras-activation). T cell anergy can also result upon stimulation with antigen in the absence of co-stimulation, resulting in the cell becoming refractory to subsequent activation by the antigen even in the context of costimulation. The unresponsive state can often be overriden by the presence of Interleukin-2. Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (costimulatory) pathways.

“Enhancing T-cellfunction” means to induce, cause or stimulate a T-cell to have a sustained or amplified biological function, or renew or reactivate exhausted or inactive T-cells. Examples of enhancing T-cell function include: increased secretion of γ-interferon from CD8⁺ T-cells, increased proliferation, increased antigen responsiveness (e.g., viral, pathogen, or tumor clearance) relative to such levels before the intervention. In one embodiment, the level of enhancement is as least 50%, alternatively 60%, 70%, 80%, 90%, 100%, 120%, 150%, 200%. The manner of measuring this enhancement is known to one of ordinary skill in the art.

A “T cell dysfunctional disorder” is a disorder or condition of T-cells characterized by decreased responsiveness to antigenic stimulation (e.g., against a tumor expressing an immunogen). In some embodiments, a T-cell dysfunctional disorder is one in which T-cells are anergic or have decreased ability to secrete cytokines, proliferate, or execute cytolytic activity. In a specific aspect, the decreased responsiveness results in ineffective control of a tumor expressing an immunogen. Examples of T cell dysfunctional disorders characterized by T-cell dysfunction include tumor immunity and cancer.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but not limited to treatment with a TIGIT inhibitor (e.g., anti-TIGIT antibodies).

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0×length of the treatment duration.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

A “VH subgroup III consensus framework” comprises the consensus sequence obtained from the amino acid sequences in variable heavy subgroup III of Kabat et al., supra. In one embodiment, the VH subgroup III consensus framework amino acid sequence comprises at least a portion or all of each of the following sequences:

(SEQ ID NO: 19)
EVQLVESGGGLVQPGGSLRLSCAAS,
(HC-FR1)
(SEQ ID NO: 20)
WVRQAPGKGLEWV,
(HC-FR2),
(HC-FR3, SEQ ID NO: 21)
RFTISADTSKNTAYLQMNSLRAEDTAVYYCAR,
(SEQ ID NO: 22)
WGQGTLVTVSA.
(HC-FR4),

A “VL kappa I consensus framework” comprises the consensus sequence obtained from the amino acid sequences in variable light kappa subgroup I of Kabat et al., supra. In one embodiment, the VH subgroup I consensus framework amino acid sequence comprises at least a portion or all of each of the following sequences: DIQMTQSPSSLSASVGDRVTITC (LC-FR1) (SEQ ID NO:23), WYQQKPGKAPKLLIY (LC-FR2) (SEQ ID NO:24), GVPSRFSGSGSGTDFTLTISSLQPEDFATYYC (LC-FR3)(SEQ ID NO:25), FGQGTKVEIKR (LC-FR4)(SEQ ID NO:26).

An “amino-acid modification” at a specified position, e.g. of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 M, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2 (including IgG2A and IgG2B), IgG-3, or IgG-4 subtypes, IgA (including IgA-1 and IgA-2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995. Immunoadhesin combinations of Ig Fc and ECD of cell surface receptors are sometimes termed soluble receptors.

A “fusion protein” and a “fusion polypeptide” refer to a polypeptide having two portions covalently linked together, where each of the portions is a polypeptide having a different property. The property may be a biological property, such as activity in vitro or in vivo. The property may also be simple chemical or physical property, such as binding to a target molecule, catalysis of a reaction, etc. The two portions may be linked directly by a single peptide bond or through a peptide linker but are in reading frame with each other.

A “blocking” antibody or an “antagonist” antibody is one that inhibits or reduces a biological activity of the antigen it binds. In some embodiments, blocking antibodies or antagonist antibodies substantially or completely inhibit the expression or biological activity of the antigen. For example, the anti-TIGIT antibodies or antigen-binding fragments thereof of the present disclosure may inhibit TIGIT expression, block the interaction of TIGIT with PVR, block the interaction of TIGIT with PVRL2, block the interaction of TIGIT with PVRL3, inhibit and/or block the intracellular signaling mediated by TIGIT binding to PVR, inhibit and/or block the intracellular signaling mediated by TIGIT binding to PVRL2, and/or inhibit and/or block the intracellular signaling mediated by TIGIT binding to PVRL3.

An “agonist” or activating antibody is one that enhances or initiates signaling by the antigen to which it binds. In some embodiments, agonist antibodies cause or activate signaling without the presence of the natural ligand.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3 and IgG4.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FeyRII, and FeyRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daeron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

As used herein, the term “treatment” or “treating” refers to clinical intervention designed to alter the natural course of the individual or cell being treated during the course of clinical pathology, e.g., cancer or tumor immunity. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying progression of a disease” means to defer, hinder, slow, retard, stabilize, and/or postpone development of the disease (such as cancer or tumor immunity). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “cancer” and “cancerous” refer to or describe the physiological condition in mammals that is typically characterized by unregulated cell growth. Included in this definition are benign and malignant cancers as well as dormant tumors or micrometastatses. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

An “effective amount” is at least the minimum concentration required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual. The term “in combination with” may be used interchangeably herein.

As used herein, the terms “individial” and “subject” may be used interchangeably and refer to a mammal, including, but not limited to, a human or non-human mammal, such as a bovine, equine, canine, ovine, or feline. Preferably, the individual or subject is a human. Patients are also individuals or subjects herein.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

A “chemotherapeutic agent” is a chemical compound useful in the treatment of cancer. Non-limiting examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaIl (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

A “chemotherapeutic agent” also includes, without limitation, anti-hormonal agents that act to regulate, reduce, block, or inhibit the effects of hormones that can promote the growth of cancer, and are often in the form of systemic, or whole-body treatment. They may be hormones themselves. Non-limiting examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1α, IL-2, IL-3, IL-4, IL-5, IL-6, IL-7, IL-8, IL-9, IL-10, IL-11, IL-12, IL-13, IL-15, IL-17A-F, IL-18 to IL-29 (such as IL-23), IL-31, including PROLEUKIN® rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The phrase “pharmaceutically acceptable” indicates that the substance or composition must be compatible chemically and/or toxicologically, with the other ingredients comprising a formulation, and/or the mammal being treated therewith.

As used herein and in the appended claims, the singular forms “a,” “or,” and “the” include plural referents unless the context clearly dictates otherwise.

Reference to “about” a value or parameter herein includes (and describes) variations that are directed to that value or parameter per se. For example, description referring to “about X” includes description of “X”.

It is understood that aspects and variations of the invention described herein include “consisting” and/or “consisting essentially of” aspects and variations.

### III. Methods

In one aspect, provided herein are methods for treating or delaying progression of cancer in an individual comprising administering to the individual an effective amount of an agent that decreases or inhibits TIGIT expression and/or activity and an anti-cancer agent and/or an anti-cancer therapy.

In another aspect, provided herein are methods for reducing or inhibiting cancer relapse or cancer progression in an individual comprising administering to the individual an effective amount of an agent that decreases or inhibits TIGIT expression and/or activity and an anti-cancer agent and/or an anti-cancer therapy.

In another aspect, provided herein are methods for treating or delaying progression of tumor immunity in an individual having cancer comprising administering to the individual an effective amount of an agent that decreases or inhibits TIGIT expression and/or activity and an anti-cancer agent and/or an anti-cancer therapy.

In another aspect, provided herein are methods for increasing, enhancing or stimulating an immune response or function in an individual having cancer comprising administering to the individual an effective amount of an agent that decreases or inhibits TIGIT expression and/or activity and an anti-cancer agent and/or an anti-cancer therapy.

In certain embodiments the anti-cancer agent may be one or more anti-cancer agents of the present disclosure. For example, the one or more anti-cancer agents may be two or more, three or more, four or more, five or more, six or more, seven or more, eight or more, nine or more, or ten or more anti-cancer agents of the present disclosure. It is to be understood that the one or more anti-cancer agents may refer to one or more anti-cancer agents that are from the same group of anti-cancer agents of the present disclosure (e.g., one or more chemotherapeutic or growth inhibitory agents of the present disclosure, one or more targeted therapeutic agents, one or more T cells expressing a chimeric antigen receptor, one or more antibodies or antigen-binding fragments thereof, one or more antibody-drug conjugates, one or more angiogenesis inhibitors, one or more antineoplastic agents, one or more cancer vaccines, and one or more adjuvants). Alternatively, the one or more anti-cancer agents may refer to one or more anti-cancer agents, where each of the one or more anti-cancer agents are from different groups of anti-cancer agents of the present disclosure (e.g., a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, and an adjuvant). In certain embodiments the anti-cancer therapy may be one or more anti-cancer therapies of the present disclosure. For example, the one or more anti-cancer therapies may be two or more, three or more, four or more, five or more, six or more, seven or more, eight or more, nine or more, or ten or more anti-cancer therapies of the present disclosure.

The methods of this invention may find use in treating conditions where enhanced immunogenicity is desired such as increasing tumor immunogenicity for the treatment of cancer or T cell dysfunctional disorders. A variety of cancers may be treated, or their progression may be delayed, by these methods.

In some embodiments, the individual has non-small cell lung cancer. The non-small cell lung cancer may be at early stage or at late stage. In some embodiments, the individual has small cell lung cancer. The small cell lung cancer may be at early stage or at late stage. In some embodiments, the individual has renal cell cancer. The renal cell cancer may be at early stage or at late stage. In some embodiments, the individual has colorectal cancer. The colorectal cancer may be at early stage or late stage. In some embodiments, the individual has ovarian cancer. The ovarian cancer may be at early stage or at late stage. In some embodiments, the individual has breast cancer. The breast cancer may be at early stage or at late stage. In some embodiments, the individual has pancreatic cancer. The pancreatic cancer may be at early stage or at late stage. In some embodiments, the individual has gastric carcinoma. The gastric carcinoma may be at early stage or at late stage. In some embodiments, the individual has bladder cancer. The bladder cancer may be at early stage or at late stage. In some embodiments, the individual has esophageal cancer. The esophageal cancer may be at early stage or at late stage. In some embodiments, the individual has mesothelioma. The mesothelioma may be at early stage or at late stage. In some embodiments, the individual has melanoma. The melanoma may be at early stage or at late stage. In some embodiments, the individual has head and neck cancer. The head and neck cancer may be at early stage or at late stage. In some embodiments, the individual has thyroid cancer. The thyroid cancer may be at early stage or at late stage. In some embodiments, the individual has sarcoma. The sarcoma may be at early stage or late stage. In some embodiments, the individual has prostate cancer. The prostate cancer may be at early stage or at late stage. In some embodiments, the individual has glioblastoma. The glioblastoma may be at early stage or at late stage. In some embodiments, the individual has cervical cancer. The cervical cancer may be at early stage or at late stage. In some embodiments, the individual has thymic carcinoma. The thymic carcinoma may be at early stage or at late stage. In some embodiments, the individual has leukemia. The leukemia may be at early stage or at late stage. In some embodiments, the individual has lymphomas. The lymphoma may be at early stage or at late stage. In some embodiments, the individual has myelomas. The myelomas may be at early stage or at late stage. In some embodiments, the individual has mycosis fungoides. The mycosis fungoides may be at early stage or at late stage. In some embodiments, the individual has merkel cell cancer. The merkel cell cancer may be at early stage or at late stage. In some embodiments, the individual has hematologic malignancies. The hematological malignancies may be early stage or late stage. In some embodiments, the individual is a human.

In some embodiments of the methods of the present disclosure, the cancer has elevated levels of T cell infiltration. As used herein, T cell infiltration of a cancer may refer to the presence of T cells, such as tumor-infiltrating lymphocytes (TILs), within or otherwise associated with the cancer tissue. It is known in the art that T cell infiltration may be associated with improved clinical outcome in certain cancers (see, e.g., Zhang et al., N. Engl. J. Med. 348(3):203-213 (2003)).

However, T cell exhaustion is also a major immunological feature of cancer, with many tumor-infiltrating lymphocytes (TILs) expressing high levels of inhibitory co-receptors and lacking the capacity to produce effector cytokines (Wherry, E. J. Nature immunology 12: 492-499 (2011); Rabinovich, G. A., et al., Annual review of immunology 25:267-296 (2007)). In some embodiments of the methods of the present disclosure, the individual has a T cell dysfunctional disorder. In some embodiments of the methods of the present disclosure, the T cell dysfunctional disorder is characterized by T cell anergy or decreased ability to secrete cytokines, proliferate or execute cytolytic activity. In some embodiments of the methods of the present disclosure, the T cell dysfunctional disorder is characterized by T cell exhaustion. In some embodiments of the methods of the present disclosure, the T cells are CD4+ and CD8+ T cells.

In some embodiments of the methods of the present disclosure, activated CD4 and/or CD8 T cells in the individual are characterized by γ-IFN⁺ producing CD4 and/or CD8 T cells and/or enhanced cytolytic activity relative to prior to the administration of the combination. γ-IFN⁺ may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells.

In some embodiments of the methods of the present disclosure, CD4 and/or CD8 T cells exhibit increased release of cytokines selected from the group consisting of IFN-γ, TNF-α and interleukins. Cytokine release may be measured by any means known in the art, e.g., using Western blot, ELISA, or immunohistochemical assays to detect the presence of released cytokines in a sample containing CD4 and/or CD8 T cells.

In some embodiments of the methods of the present disclosure, the CD4 and/or CD8 T cells are effector memory T cells. In some embodiments of the methods of the present disclosure, the CD4 and/or CD8 effector memory T cells are characterized by having the expression of CD44^{high} CD62L^{Low}. Expression of CD44^{high} CD62L^{Low} may be detected by any means known in the art, e.g., by preparing single cell suspensions of tissue (e.g., a cancer tissue) and performing surface staining and flow cytometry using commercial antibodies against CD44 and CD62L.

Agents that Decrease or Inhibit TIGIT Expression and/or Activity

Certain aspects of the present disclosure relate to agents that decrease or inhibit TIGIT expression and/or activity. As used herein, “TIGIT” may refer to any polypeptide or homolog thereof characterized or predicted to function as a T Cell Immunoreceptor with Ig and ITIM Domains polypeptide. A non-limiting example of a TIGIT polypeptide is any polypeptide encoded by the human gene represented by NCBI Gene ID No. 201633, such as a polypeptide having the sequence described by NCBI RefSeq No. NP_776160. Additional description of TIGIT polypeptides and their biological activity may be found in Yu et al., Nat. Immunol. 10:48-57 (2009), US patent publication no. US20040121370, and US patent publication no. US20130251720.

Provided herein is a method for treatment or delaying progression of cancer in an individual comprising administering to the individual an effective amount of an agent that decreases or inhibits TIGIT expression and/or activity and an anti-cancer agent and/or an anti-cancer therapy. Provided herein is also a method for reducing or inhibiting cancer relapse or cancer progression in an individual comprising administering to the individual an effective amount of an agent that decreases or inhibits TIGIT expression and/or activity and an anti-cancer agent and/or an anti-cancer therapy. Provided herein is also a method for treating or delaying progression of tumor immunity in an individual having cancer comprising administering to the individual an effective amount of an agent that decreases or inhibits TIGIT expression and/or activity and an anti-cancer agent and/or an anti-cancer therapy. Provided herein is also a method for increasing, enhancing or stimulating an immune response or function in an individual having cancer comprising administering to the individual an effective amount of an agent that decreases or inhibits TIGIT expression and/or activity and an anti-cancer agent and/or an anti-cancer therapy.

In some embodiments, an agent that decreases or inhibits TIGIT expression and/or activity includes an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof.

In some embodiments, the antagonist of TIGIT expression and/or activity includes a small molecule inhibitor, an inhibitory antibody or antigen-binding fragment thereof, an aptamer, an inhibitory nucleic acid, and an inhibitory polypeptide.

In some embodiments, the antagonist of PVR expression and/or activity includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVR includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVRL2 includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVRL3 includes a small molecule inhibitor, an inhibitory antibody or antigen-binding fragment thereof, an aptamer, an inhibitory nucleic acid, and an inhibitory polypeptide

In some embodiments, the agent that inhibits and/or blocks the intracellular signaling mediated by TIGIT binding to PVR includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the intracellular signaling mediated by TIGIT binding to PVRL2 includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the intracellular signaling mediated by TIGIT binding to PVRL3 includes a small molecule inhibitor, an inhibitory antibody or antigen-binding fragment thereof, an aptamer, an inhibitory nucleic acid, and an inhibitory polypeptide.

In some embodiments, the antagonist of TIGIT expression and/or activity is an inhibitory nucleic acid selected from an antisense polynucleotide, an interfering RNA, a catalytic RNA, and an RNA-DNA chimera.

In some embodiments, the antagonist of TIGIT expression and/or activity is an anti-TIGIT antibody or antigen-binding fragment thereof.

The anti-TIGIT antibodies useful in this invention, including compositions containing such antibodies, such as those described in WO 2009/126688, may be used in combination with anti-cancer agents.

### Anti-TIGIT Antibodies

The present invention provides anti-TIGIT antibodies. Exemplary antibodies include polyclonal, monoclonal, humanized, bispecific, and heteroconjugate antibodies. It will be understood by one of ordinary skill in the art that the invention also provides antibodies against other polypeptides (i.e., anti-PVR antibodies) and that any of the description herein drawn specifically to the method of creation, production, varieties, use or other aspects of anti-TIGIT antibodies will also be applicable to antibodies specific for other non-TIGIT polypeptides.

### Polyclonal Antibodies

The anti-TIGIT antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the TIGIT polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

### Monoclonal Antibodies

The anti-TIGIT antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the TIGIT polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

### Human and Humanized Antibodies

The anti-TIGIT antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

### Bispecific Antibodies

Bispecific antibodies are monoclonal, preferably human or humanized, antibodies that have binding specificities for at least two different antigens. In the present case, one of the binding specificities is for TIGIT, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)2 bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. As one nonlimiting example, trispecific antibodies can be prepared. See, e.g., Tutt et al., J. Immunol. 147:60 (1991).

Exemplary bispecific antibodies may bind to two different epitopes on a given TIGIT polypeptide herein. Alternatively, an anti-TIGIT polypeptide arm may be combined with an arm which binds to a triggering molecule on a leukocyte such as a T-cell receptor molecule (e.g., CD3), or Fc receptors for IgG (FcγR), such as FcγRI (CD64), FcγRII (CD32) and FcγRIII (CD16) so as to focus cellular defense mechanisms to the cell expressing the particular TIGIT polypeptide. Bispecific antibodies may also be used to localize cytotoxic agents to cells which express a particular TIGIT polypeptide. These antibodies possess a TIGIT-binding arm and an arm which binds a cytotoxic agent or a radionuclide chelator, such as EOTUBE, DPTA, DOTA, or TETA. Another bispecific antibody of interest binds the TIGIT polypeptide and further binds tissue factor (TF).

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced complement lysis and ADCC capabilities. See Stevenson et al., Anti-Cancer Drug Design, 3: 219-230 (1989).

In some embodiments, anti-TIGIT antibodies were generated which were hamster-anti-mouse antibodies. Two antibodies, 10A7 and 1F4, also specifically bound to human TIGIT. The amino acid sequences of the light and heavy chains of the 10A7 antibody were determined using standard techniques. The light chain sequence of this antibody is: DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGT KLEIKR (SEQ ID NO:13) and the heavy chain sequence of this antibody is: EVQLVESGGGLTQPGKSLKLSCEASGFTFSSFTMHWVRQSPGKGLEWVAFIRSGSGIV FYADAVRGRFTISRDNAKNLLFLQMNDLKSEDTAMYYCARRPLGHNTFDSWGQGTLV TVSS (SEQ ID NO:15), where the hypervariable regions (HVRs) of each chain are represented by bold text. Thus, HVR1 of the 10A7 light chain has the sequence KSSQSLYYSGVKENLLA (SEQ ID NO:1), HVR2 of the 10A7 light chain has the sequence ASIRFT (SEQ ID NO:2), and HVR3 of the 10A7 light chain has the sequence QQGINNPLT (SEQ ID NO:3). HVR1 of the 10A7 heavy chain has the sequence GFTFSSFTMH (SEQ ID NO:4), HVR2 of the 10A7 heavy chain has the sequence FIRSGSGIVFYADAVRG (SEQ ID NO:5), and HVR3 of the 10A7 heavy chain has the sequence RPLGHNTFDS (SEQ ID NO:6).

The amino acid sequences of the light and heavy chains of the 1F4 antibody were also determined. The light chain sequence of this antibody is: DVVLTQTPLSLSVSFGDQVSISCRSSQSLVNSYGNTFLSWYLHKPGQSPQLLIFGISNRF SGVPDRFSGSGSGTDFTLKISTIKPEDLGMYYCLQGTHQPPTFGPGTKLEVK (SEQ ID NO:14) and the heavy chain sequence of this antibody is: EVQLQQSGPELVKPGTSMKISCKASGYSFTGHLMNWVKQSHGKNLEWIGLIIPYNGGT SYNQKFKGKATLTVDKSSSTAYMELLSLTSDDSAVYFCSRGLRGFYAMDYWGQGTSV TVSS (SEQ ID NO:16), where the hypervariable regions (HVRs) of each chain are represented by bold text. Thus, HVR1 of the 1F4 light chain has the sequence RSSQSLVNSYGNTFLS (SEQ ID NO:7), HVR2 of the 1F4 light chain has the sequence GISNRFS (SEQ ID NO:8), and HVR3 of the 1F4 light chain has the sequence LQGTHQPPT (SEQ ID NO:9). HVR1 of the 1F4 heavy chain has the sequence GYSFTGHLMN (SEQ ID NO:10), HVR2 of the 1F4 heavy chain has the sequence LIIPYNGGTSYNQKFKG (SEQ ID NO:11), and HVR3 of the 1F4 heavy chain has the sequence GLRGFYAMDY (SEQ ID NO:12).

The nucleotide sequence encoding the 1F4 light chain was determined to be GATGTTGTGTTGACTCAAACTCCACTCTCCCTGTCTGTCAGCTTTGGAGATCAAGTTT CTATCTCTTGCAGGTCTAGTCAGAGTCTTGTAAACAGTTATGGGAACACCTTTTTGTC TTGGTACCTGCACAAGCCTGGCCAGTCTCCACAGCTCCTCATCTTTGGGATTTCCAA CAGATTTTCTGGGGTGCCAGACAGGTTCAGTGGCAGTGGTTCAGGGACAGATTTCAC ACTCAAGATCAGCACAATAAAGCCTGAGGACTTGGGAATGTATTACTGCTTACAAG GTACGCATCAGCCTCCCACGTTCGGTCCTGGGACCAAGCTGGAGGTGAAA (SEQ ID NO:17) and the nucleotide sequence encoding the 1F4 heavy chain was determined to be GAGGTCCAGCTGCAACAGTCTGGACCTGAGCTGGTGAAGCCTGGAACTTCAATGAA GATATCCTGCAAGGCTTCTGGTTACTCATTCACTGGCCATCTTATGAACTGGGTGAA GCAGAGCCATGGAAAGAACCTTGAGTGGATTGGACTTATTATTCCTTACAATGGTGG TACAAGCTATAACCAGAAGTTCAAGGGCAAGGCCACATTGACTGTAGACAAGTCAT CCAGCACAGCCTACATGGAGCTCCTCAGTCTGACTTCTGATGACTCTGCAGTCTATTT CTGTTCAAGAGGCCTTAGGGGCTTCTATGCTATGGACTACTGGGGTCAAGGAACCTC AGTCACCGTCTCCTCA (SEQ ID NO:18).

In some embodiments, the anti-TIGIT antibody or antigen-binding fragment thereof comprises at least one HVR comprising an amino acid sequence selected from the amino acid sequences set forth in KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6) or RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

In some embodiments, the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14).

In some embodiments, the anti-TIGIT antibody or antigen-binding fragment thereof, wherein the antibody heavy chain comprises the amino acid sequence set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO:16).

In some embodiments, the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14) and the antibody heavy chain comprises the amino acid sequence set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO:16).

In some embodiments, the anti-TIGIT antibody or antigen-binding fragment thereof, wherein the antibody is selected from a humanized antibody, a chimeric antibody, a bispecific antibody, a heteroconjugate antibody, and an immunotoxin.

In some embodiments, the anti-TIGIT antibody or antigen-binding fragment thereof comprises at least one HVR is at least 90% identical to an HVR set forth in any of KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6) or RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

In some embodiments, the anti-TIGIT antibody or fragment thereof comprises the light chain and/or heavy chain comprising amino acid sequences at least 90% identical to the amino acid sequences set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14), or EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO:16), respectively.

### Anti-Cancer Agents and Anti-Cancer Therapies

Certain aspects of the present disclosure relate to anti-cancer agents and anti-cancer therapies. Without wishing to be bound to theory, it is thought that, because of the properties of TIGIT (see, e.g., US patent publication no. US20040121370 and US patent publication no. US20130251720), an agent that decreases or inhibits TIGIT expression and/or activity may enhance the anti-cancer effect of an anti-cancer agent and/or anti-cancer therapy, e.g., by treating or delaying progression of cancer; reducing or inhibiting cancer relapse or cancer progression; treating or delaying progression of tumor immunity; and/or increasing, enhancing or stimulating an immune response or function in an individual having cancer. As will be recognized by one of skill in the art, the anti-cancer agents and anti-cancer therapies described herein may be used individually or in conjunction. As disclosed herein, an agent that decreases or inhibits TIGIT expression and/or activity of the present disclosure may be administered with an anti-cancer agent of the present disclosure, with an anti-cancer therapy of the present disclosure, or with an anti-cancer agent of the present disclosure and an anti-cancer therapy of the present disclosure.

In some embodiments, an agent that decreases or inhibits TIGIT expression and/or activity may be administered in conjunction with an anti-cancer therapy. An anti-cancer therapy of the present disclosure may include radiation therapy, surgery, chemotherapy, gene therapy (e.g., a gene therapy vaccine such as ALLOVECTIN®, LEUVECTIN®, and VAXID®), DNA therapy, viral therapy, RNA therapy, immunotherapy, bone marrow transplantation, nanotherapy, monoclonal antibody therapy, or combinations thereof. The anti-cancer therapy may be in the form of an adjuvant or neoadjuvant therapy. In some embodiments, the anti-cancer therapy is the administration of side-effect limiting agents (e.g., agents intended to lessen the occurrence and/or severity of side effects of treatment, such as anti-nausea agents, etc.). In some embodiments, the anti-cancer therapy is radiation therapy. In some embodiments, the anti-cancer therapy is surgery. In some embodiments, the anti-cancer therapy may be one or more of the chemotherapeutic agents described herein. Any of these therapies may be administered in conjunction with an agent that decreases or inhibits TIGIT expression and/or activity of the present disclosure.

In some embodiments, the anti-cancer agent is a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof.

In some embodiments, the anti-cancer agent is a chemotherapeutic or growth inhibitory agent. For example, a chemotherapeutic or growth inhibitory agent may include an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof.

Examples of chemotherapeutic agents may include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

In some embodiments, a chemotherapeutic agent may include alkylating agents (including monofunctional and bifunctional alkylators) such as thiotepa, CYTOXAN® cyclosphosphamide, nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; temozolomide; and pharmaceutically acceptable salts, acids and derivatives of any of the above.

In some embodiments, a chemotherapeutic agent may include anthracyclines such as daunorubicin, doxorubicin, epirubicin, idarubicin, mitoxantrone, valrubicin, and pharmaceutically acceptable salts, acids and derivatives of any of the above.

In some embodiments, a chemotherapeutic agent may include an anti-hormonal agent such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); and pharmaceutically acceptable salts, acids and derivatives of any of the above.

In some embodiments, a chemotherapeutic agent may include an aromatase inhibitor that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); and pharmaceutically acceptable salts, acids and derivatives of any of the above.

In some embodiments, a chemotherapeutic agent may include an anti-androgen such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); and pharmaceutically acceptable salts, acids and derivatives of any of the above.

In some embodiments, a chemotherapeutic agent may include a protein kinase inhibitors, lipid kinase inhibitor, or an antisense oligonucleotide, particularly those which inhibit expression of genes in signaling pathways implicated in aberrant cell proliferation, such as, for example, PKC-alpha, Ralf and H-Ras.

In some embodiments, a chemotherapeutic agent may include a ribozyme such as VEGF expression inhibitors (e.g., ANGIOZYME®) and HER2 expression inhibitors.

In some embodiments, a chemotherapeutic agent may include a cytotoxic agent or antitumor antibiotic, such as dactinomycin, actinomycin, bleomycins, plicamycin, mitomycins such as mitomycin C, and pharmaceutically acceptable salts, acids and derivatives of any of the above.

In some embodiments, a chemotherapeutic agent may include a proteasome inhibitor such as bortezomib (VELCADE®, Millennium Pharm.), epoxomicins such as carfilzomib (KYPROLIS®, Onyx Pharm.), marizomib (NPI-0052), MLN2238, CEP-18770, oprozomib, and pharmaceutically acceptable salts, acids and derivatives of any of the above.

In some embodiments, a chemotherapeutic agent may include an anti-microtubule agent such as Vinca alkaloids, including vincristine, vinblastine, vindesine, and vinorelbine; taxanes, including paclitaxel and docetaxel; podophyllotoxin; and pharmaceutically acceptable salts, acids and derivatives of any of the above.

In some embodiments, a chemotherapeutic agent may include an “EGFR antagonist,” which refers to a compound that binds to or otherwise interacts directly with EGFR and prevents or reduces its signaling activity, and is alternatively referred to as an “EGFR i.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

In some embodiments, a chemotherapeutic agent may include a tyrosine kinase inhibitor, including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

In some embodiments, a chemotherapeutic agent may include a retinoid such as retinoic acid and pharmaceutically acceptable salts, acids and derivatives of any of the above.

In some embodiments, a chemotherapeutic agent may include an anti-metabolite. Examples of anti-metabolites may include folic acid analogs and antifolates such as denopterin, methotrexate, pteropterin, trimetrexate; purine analogs such as fludarabine, 6-mercaptopurine, thiamiprine, thioguanine; pyrimidine analogs such as 5-fluorouracil (5-FU), ancitabine, azacitidine, 6-azauridine, carmofur, cytarabine, dideoxyuridine, doxifluridine, enocitabine, floxuridine; nucleoside analogs; and nucleotide analogs.

In some embodiments, a chemotherapeutic agent may include a topoisomerase inhibitor. Examples of topoisomerase inhibitors may include a topoisomerase 1 inhibitor such as LURTOTECAN® and ABARELIX® rmRH; a topoisomerase II inhibitor such as doxorubicin, epirubicin, etoposide, and bleomycin; and topoisomerase inhibitor RFS 2000.

In some embodiments, a chemotherapeutic agent may include a histone deacetylase inhibitor such as vorinostat, romidepsin, belinostat, mocetinostat, valproic acid, panobinostate, and pharmaceutically acceptable salts, acids and derivatives of any of the above.

Chemotherapeutic agents may also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTal/02 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents may also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

A “growth inhibitory agent” when used herein refers to a compound or composition which inhibits growth of a cell either in vitro or in vivo. As such, one of skill in the art will appreciate that many agents (such as many of those described above) may be categorized as both chemotherapeutic agents and growth inhibitory agents. In one embodiment, the growth inhibitory agent is growth inhibitory antibody that prevents or reduces proliferation of a cell expressing an antigen to which the antibody binds. In another embodiment, the growth inhibitory agent may be one which significantly reduces the percentage of cells in S phase. Examples of growth inhibitory agents may include agents that block cell cycle progression (at a place other than S phase), such as agents that induce G1 arrest and M-phase arrest. Classical M-phase blockers include the vinca alkaloids (vincristine and vinblastine), taxanes, and topoisomerase II inhibitors such as doxorubicin, epirubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

In some embodiments, the anti-cancer agent is a targeted therapeutic agent. For example, a targeted therapeutic agent may include a B-raf inhibitor, a MEK inhibitor, a K-ras inhibitor, a c-Met inhibitor, an Alk inhibitor, a phosphatidylinositol 3-kinase inhibitor, an Akt inhibitor, an mTOR inhibitor, a dual phosphatidylinositol 3-kinase/mTOR inhibitor, and combinations thereof. As used herein, the term “inhibitor” is used in the broadest sense to encompass any small molecule, protein, or other macromolecule that interferes with a biological activity of its target.

In some embodiments, a targeted therapeutic agent may include a B-Raf inhibitor such as vemurafenib (also known as Zelboraf@), dabrafenib (also known as Tafinlar@), and erlotinib (also known as Tarceva®); a MEK inhibitor, such as an inhibitor of MEK1 (also known as MAP2K1) or MEK2 (also known as MAP2K2), cobimetinib (also known as GDC-0973 or XL-518), and trametinib (also known as Mekinist®); a K-Ras inhibitor; a c-Met inhibitor such as onartuzumab (also known as MetMAb); an Alk inhibitor such as AF802 (also known as CH5424802 or alectinib); a phosphatidylinositol 3-kinase (PI3K) inhibitor such as idelalisib (also known as GS-1101 or CAL-101), BKM120, and perifosine (also known as KRX-0401); an Akt inhibitor such as GSK690693, MK2206, and GDC-0941; an mTOR inhibitor such as sirolimus (also known as rapamycin), temsirolimus (also known as CCI-779 or Torisel@), everolimus (also known as RAD001), ridaforolimus (also known as AP-23573, MK-8669, or deforolimus), OSI-027, AZD8055, and INK128; and a dual phosphatidylinositol 3-kinase (PI3K)/mTOR inhibitor such as XL765, GDC-0980, BEZ235 (also known as NVP-BEZ235), BGT226, GSK2126458, PF-04691502, and PF-05212384 (also known as PKI-587).

In some embodiments, the anti-cancer agent is a T cell expressing a chimeric antigen receptor. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. For example, a T cell expressing a chimeric antigen receptor may express a dominant-negative TGF beta receptor, e.g, a dominant-negative TGF beta type II receptor. Examples of a treatment using a T cell expressing a chimeric antigen receptor and a dominant-negative TGF beta receptor include the HERCREEM protocol (see, e.g., ClinicalTrials.gov Identifier NCT00889954).

In some embodiments, the anti-cancer agent is an antibody or antigen-binding fragment thereof. For example, an antibody or antigen-binding fragment thereof may include alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), the antibody drug conjugate, gemtuzumab ozogamicin (MYLOTARG®, Wyeth), and combinations thereof. Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, clivatuzumab, daclizumab, eculizumab, efalizumab, epratuzumab, erlizumab, felvizumab, fontolizumab, gemtuzumab ozogamicin, inotuzumab ozogamicin, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pertuzumab, pexelizumab, ralivizumab, ranibizumab, reslivizumab, reslizumab, resyvizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, anti-IL-12 (e.g., ABT-874/J695, Wyeth Research and Abbott Laboratories, which is a recombinant exclusively human-sequence, full-length IgG₁ λ antibody genetically modified to recognize IL-12 p40 protein), anti-IL-17 (e.g., MCAF5352A or RG7624), and combinations thereof.

In some embodiments, the anti-cancer agent is an antibody or antigen-binding fragment thereof that specifically binds to a target selected from CD52, VEGF-A, EGFR, CD20, HER2, HLA-DRB, CD62L, IL-6R, amyloid beta, CD44, CanAg, CD4, TNF alpha, IL-2, CD25, complement C5, CD11a, CD22, CD18, respiratory syncytial virus F, interferon gamma, CD33, CEACAM5, IL-5, integrin alpha 4, IgE, IL-4, IL-5, CD154, FAP, CD2, MUC-1, AFP, integrin alIbP3, ClfA, IL6R, CD40L, EpCAM, Shiga-like toxin II, IL-12, IL-23, IL-17, and CD3. In some embodiments, an antibody or antigen-binding fragment thereof that specifically binds to IL-17 (such as anti-IL-17 as described above) may include an antibody or antigen-binding fragment thereof that specifically binds to IL-17A, IL-17B, IL-17C, IL-17D, IL-17E, IL-17F, and combinations thereof.

In some embodiments, the anti-cancer agent is an antibody-drug conjugate. For example, an antibody-drug conjugate may include mertansine or monomethyl auristatin E (MMAE), such as an anti-NaPi2b antibody-MMAE conjugate (also known as DNIB0600A or RG7599), trastuzumab emtansine (also known as T-DM1, ado-trastuzumab emtansine, or KADCYLA®, Genentech), DMUC5754A, bivatuzumab mertansine or cantuzumab mertansine, and an antibody-drug conjugate targeting the endothelin B receptor (EDNBR), e.g., an antibody directed against EDNBR conjugated with MMAE. For example, an antibody-drug conjugate may also include a calicheamicin or an esperamicin (e.g., calicheamicin k or esperamicin A1), such as gemtuzumab ozogamicin (MYLOTARG®, Wyeth) or inotuzumab ozogamicin. For example, an antibody-drug conjugate may also include a radioisotope chelator, e.g., a tetraxetan, such as with tacatuzumab tetraxetan or clivatuzumab tetraxetan, or a tiuxetan, as with ibritumomab tiuxetan (ZEVALIN®, Spectrum Pharma.). The term “antibody” as it relates to an antibody-drug conjugate of the present disclosure is used in the broadest sense and specifically covers monoclonal antibodies, polyclonal antibodies, multispecific antibodies (e.g., bispecific antibodies) formed from at least two intact antibodies, and antibody fragments (e.g., a Fab fragment, scFv, minibody, diabody, scFv multimer, or bispecific antibody fragment) so long as they exhibit the desired biological activity, i.e., specific binding to an antigen and the ability to be conjugated to a drug.

In some embodiments, the anti-cancer agent is an angiogenesis inhibitor. For example, an angiogenesis inhibitor may include a VEGF antagonist, e.g., an antagonist of VEGF-A such as bevacizumab (also known as AVASTIN®, Genentech); and an angiopoietin 2 antagonist (also known as Ang2) such as MEDI3617. In some embodiments, the angiogenesis inhibitor may include an antibody.

In some embodiments, the anti-cancer agent is an antineoplastic agent. For example, an antineoplastic agent may include an agent targeting CSF-1R (also known as M-CSFR or CD115) such as anti-CSF-1R (also known as IMC-CS4); an interferon, e.g., interferon alpha or interferon gamma, such as Roferon-A (also known as recombinant Interferon alpha-2a); GM-CSF (also known as recombinant human granulocyte macrophage colony stimulating factor, rhu GM-CSF, sargramostim, or Leukine®); IL-2 (also known as aldesleukin or Proleukin®); IL-12; and an antibody targeting CD20 such as obinutuzumab (also known as GA101 or Gazyva®) or rituximab.

In some embodiments, the anti-cancer agent is a cancer vaccine. For example, a cancer vaccine may include a peptide cancer vaccine, which in some embodiments is a personalized peptide vaccine. In some embodiments the peptide cancer vaccine is a multivalent long peptide vaccine, a multi-peptide vaccine, a peptide cocktail vaccine, a hybrid peptide vaccine, or a peptide-pulsed dendritic cell vaccine (see, e.g., Yamada et al., Cancer Sci, 104:14-21, 2013).

In some embodiments, the anti-cancer agent is an adjuvant. Any substance that enhances an anti-cancer immune response, such as against a cancer-related antigen, or aids in the presentation of a cancer antigen to a component of the immune system may be considered an anti-cancer adjuvant of the present disclosure.

In some embodiments, the anti-cancer agent is an agent selected from a TLR agonist, e.g., Poly-ICLC (also known as Hiltonol®), LPS, MPL, or CpG ODN; tumor necrosis factor (TNF) alpha; IL-1; HMGB1; an IL-10 antagonist; an IL-4 antagonist; an IL-13 antagonist; a treatment targeting CX3CL1; a treatment targeting CXCL9; a treatment targeting CXCL10; a treatment targeting CCL5; an LFA-1 or ICAM1 agonist; and a Selectin agonist.

### IV. Kits

In another aspect, provided is a kit comprising an anti-cancer agent and a package insert comprising instructions for using the anti-cancer agent in combination with an agent that decreases or inhibits TIGIT expression and/or activity to treat or delay progression of cancer in an individual. Any of the agents that decrease or inhibit TIGIT expression and/or activity and/or anti-cancer agents described herein may be included in the kit.

In another aspect, provided is a kit comprising an anti-cancer agent and an agent that decreases or inhibits TIGIT expression and/or activity, and a package insert comprising instructions for using the anti-cancer agent and the agent that decreases or inhibits TIGIT expression and/or activity to treat or delay progression of cancer in an individual. Any of the agents that decrease or inhibit TIGIT expression and/or activity and/or anti-cancer agents described herein may be included in the kit.

In another aspect, provided is a kit comprising an agent that decreases or inhibits TIGIT expression and/or activity and a package insert comprising instructions for using the agent that decreases or inhibits TIGIT expression and/or activity in combination with an anti-cancer agent and/or an anti-cancer therapy to treat or delay progression of cancer in an individual. Any of the agents that decrease or inhibit TIGIT expression and/or activity and anti-cancer agents or anti-cancer therapies described herein may be included in the kit.

In another aspect, provided is a kit comprising an anti-cancer agent and a package insert comprising instructions for using the anti-cancer agent in combination with an agent that decreases or inhibits TIGIT expression and/or activity to reduce or inhibit cancer relapse or cancer progression in an individual having cancer. Any of the agents that decrease or inhibit TIGIT expression and/or activity and/or anti-cancer agents described herein may be included in the kit.

In another aspect, provided is a kit comprising an anti-cancer agent and an agent that decreases or inhibits TIGIT expression and/or activity, and a package insert comprising instructions for using the anti-cancer agent and the agent that decreases or inhibits TIGIT expression and/or activity to reduce or inhibit cancer relapse or cancer progression in an individual having cancer. Any of the agents that decrease or inhibit TIGIT expression and/or activity and/or anti-cancer agents described herein may be included in the kit.

In another aspect, provided is a kit comprising an agent that decreases or inhibits TIGIT expression and/or activity and a package insert comprising instructions for using the agent that decreases or inhibits TIGIT expression and/or activity in combination with an anti-cancer agent and/or anti-cancer therapy to reduce or inhibit cancer relapse or cancer progression in an individual having cancer. Any of the agents that decrease or inhibit TIGIT expression and/or activity and anti-cancer agents or anti-cancer therapies described herein may be included in the kit.

In another aspect, provided is a kit comprising an anti-cancer agent and a package insert comprising instructions for using the anti-cancer agent in combination with an agent that decreases or inhibits TIGIT expression and/or activity to treat or delay progression of tumor immunity in an individual having cancer. Any of the agents that decrease or inhibit TIGIT expression and/or activity and/or anti-cancer agents described herein may be included in the kit.

In another aspect, provided is a kit comprising an anti-cancer agent and an agent that decreases or inhibits TIGIT expression and/or activity, and a package insert comprising instructions for using the anti-cancer agent and the agent that decreases or inhibits TIGIT expression and/or activity to treat or delay progression of tumor immunity in an individual having cancer. Any of the agents that decrease or inhibit TIGIT expression and/or activity and/or anti-cancer agents described herein may be included in the kit.

In another aspect, provided is a kit comprising an agent that decreases or inhibits TIGIT expression and/or activity and a package insert comprising instructions for using the agent that decreases or inhibits TIGIT expression and/or activity in combination with an anti-cancer agent and/or anti-cancer therapy to treat or delay progression of tumor immunity in an individual having cancer. Any of the agents that decrease or inhibit TIGIT expression and/or activity and anti-cancer agents or anti-cancer therapies described herein may be included in the kit.

In another aspect, provided is a kit comprising an anti-cancer agent and a package insert comprising instructions for using the anti-cancer agent in combination with an agent that decreases or inhibits TIGIT expression and/or activity to increase, enhance, or stimulate an immune response or function in an individual having cancer. Any of the agents that decrease or inhibit TIGIT expression and/or activity and/or anti-cancer agents described herein may be included in the kit.

In another aspect, provided is a kit comprising an anti-cancer agent and an agent that decreases or inhibits TIGIT expression and/or activity, and a package insert comprising instructions for using the anti-cancer agent and the agent that decreases or inhibits TIGIT expression and/or activity to increase, enhance, or stimulate an immune response or function in an individual having cancer. Any of the agents that decrease or inhibit TIGIT expression and/or activity and/or anti-cancer agents described herein may be included in the kit.

In another aspect, provided is a kit comprising an agent that decreases or inhibits TIGIT expression and/or activity and a package insert comprising instructions for using the agent that decreases or inhibits TIGIT expression and/or activity in combination with an anti-cancer agent and/or anti-cancer therapy to increase, enhance, or stimulate an immune response or function in an individual having cancer. Any of the agents that decrease or inhibit TIGIT expression and/or activity and anti-cancer agents or anti-cancer therapies described herein may be included in the kit.

In some embodiments, the kit comprises a container containing one or more of the agents that decrease or inhibit TIGIT expression and/or activity and anti-cancer agents described herein. Suitable containers include, for example, bottles, vials (e.g., dual chamber vials), syringes (such as single or dual chamber syringes) and test tubes. The container may be formed from a variety of materials such as glass or plastic. In some embodiments, the kit may comprise a label (e.g., on or associated with the container) or a package insert. The label or the package insert may indicate that the compound contained therein may be useful or intended for treating or delaying progression of cancer in an individual or for enhancing immune function of an individual having cancer. The kit may further comprise other materials desirable from a commercial and user standpoint, including other buffers, diluents, filters, needles, and syringes.

All patents, patent applications, documents, and articles cited herein are herein incorporated by reference in their entireties.

What is claimed is:

1. A method for treating or delaying progression of cancer in an individual comprising administering to the individual an effective amount of an agent that decreases or inhibits TIGIT expression and/or activity and an anti-cancer agent or an anti-cancer therapy.

2. A method for reducing or inhibiting cancer relapse or cancer progression in an individual comprising administering to the individual an effective amount of an agent that decreases or inhibits TIGIT expression and/or activity and an anti-cancer agent or an anti-cancer therapy.

3. A method for treating or delaying progression of tumor immunity in an individual having cancer comprising administering to the individual an effective amount of an agent that decreases or inhibits TIGIT expression and/or activity and an anti-cancer agent or an anti-cancer therapy.

4. A method of increasing, enhancing or stimulating an immune response or function in an individual having cancer comprising administering to the individual an effective amount of an agent that decreases or inhibits TIGIT expression and/or activity and an anti-cancer agent or an anti-cancer therapy.

5. The method of any one of claims 1-4, wherein the individual has a T cell dysfunctional disorder.

6. The method of claim 5, wherein the T cell dysfunctional disorder is characterized by T cell anergy or decreased ability to secrete cytokines, proliferate or execute cytolytic activity.

7. The method of claim 5, wherein the T cell dysfunctional disorder is characterized by T cell exhaustion.

8. The method of any one of claims 5-7, wherein the T cells are CD4+ and CD8+ T cells.

9. The method of any one of claims 1-8, wherein the agent that decreases or inhibits TIGIT expression and/or activity is selected from the group consisting of an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof.

10. The method of claim 9, wherein the antagonist of TIGIT expression and/or activity is selected from the group consisting of a small molecule inhibitor, an inhibitory antibody or antigen-binding fragment thereof, an aptamer, an inhibitory nucleic acid, and an inhibitory polypeptide.

11. The method of claim 9, wherein the antagonist of PVR expression and/or activity is selected from the group consisting of a small molecule inhibitor, an inhibitory antibody or antigen-binding fragment thereof, an aptamer, an inhibitory nucleic acid, and an inhibitory polypeptide.

12. The method of claim 9, wherein the agent that inhibits and/or blocks the interaction of TIGIT with PVR is selected from the group consisting of a small molecule inhibitor, an inhibitory antibody or antigen-binding fragment thereof, an aptamer, an inhibitory nucleic acid, and an inhibitory polypeptide.

13. The method of claim 9, wherein the agent that inhibits and/or blocks the interaction of TIGIT with PVRL2 is selected from the group consisting of a small molecule inhibitor, an inhibitory antibody or antigen-binding fragment thereof, an aptamer, an inhibitory nucleic acid, and an inhibitory polypeptide.

14. The method of claim 9, wherein the agent that inhibits and/or blocks the interaction of TIGIT with PVRL3 is selected from the group consisting of a small molecule inhibitor, an inhibitory antibody or antigen-binding fragment thereof, an aptamer, an inhibitory nucleic acid, and an inhibitory polypeptide.

15. The method of claim 9, wherein the agent that inhibits and/or blocks the intracellular signaling mediated by TIGIT binding to PVR is selected from the group consisting of a small molecule inhibitor, an inhibitory antibody or antigen-binding fragment thereof, an aptamer, an inhibitory nucleic acid, and an inhibitory polypeptide.

16. The method of claim 9, wherein the agent that inhibits and/or blocks the intracellular signaling mediated by TIGIT binding to PVRL2 is selected from the group consisting of a small molecule inhibitor, an inhibitory antibody or antigen-binding fragment thereof, an aptamer, an inhibitory nucleic acid, and an inhibitory polypeptide.

17. The method of claim 9, wherein the agent that inhibits and/or blocks the intracellular signaling mediated by TIGIT binding to PVRL3 is selected from the group consisting of a small molecule inhibitor, an inhibitory antibody or antigen-binding fragment thereof, an aptamer, an inhibitory nucleic acid, and an inhibitory polypeptide.

18. The method of claim 10, wherein the inhibitory nucleic acid is selected from the group consisting of an antisense polynucleotide, an interfering RNA, a catalytic RNA, and an RNA-DNA chimera.

19. The method of claim 9, wherein the antagonist of TIGIT expression and/or activity is an anti-TIGIT antibody or antigen-binding fragment thereof.

20. The method of claim 19, wherein the anti-TIGIT antibody or antigen-binding fragment thereof is selected from the group consisting of a humanized antibody, a chimeric antibody, a bispecific antibody, a heteroconjugate antibody, and an immunotoxin.

21. The method of claim 19 or 20, wherein the anti-TIGIT antibody or antigen-binding fragment thereof comprises at least one HVR comprising an amino acid sequence selected from the amino acid sequences (1) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (2) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

22. The method of any one of claims 19-21, wherein the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14).

23. The method of any one of claims 19-22, wherein the anti-TIGIT antibody or antigen-binding fragment thereof, wherein the antibody heavy chain comprises the amino acid sequence set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO:16).

24. The method of any one of claims 19-23, wherein the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14), and the antibody heavy chain comprises the amino acid sequence set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO: 16).

25. The method of claim 19 or 20, wherein the anti-TIGIT antibody or antigen-binding fragment thereof comprises at least one HVR that is at least 90% identical to an HVR set forth in any one of (1) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (2) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

26. The method of claim 25, wherein the anti-TIGIT antibody or fragment thereof comprises the light chain comprising amino acid sequences at least 90% identical to the amino acid sequences set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14); and/or the heavy chain comprising amino acid sequences at least 90% identical to the amino acid sequences set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO:16).

27. The method of any one of the preceding claims, comprising administering to the individual an effective amount of an agent that decreases or inhibits TIGIT expression and/or activity, an anti-cancer agent, and an anti-cancer therapy.

28. The method of any one of the preceding claims, wherein the anti-cancer agent is one or more anti-cancer agents.

29. The method of claim 28, wherein the one or more anti-cancer agents are two or more anti-cancer agents.

30. The method of claim 28, wherein the one or more anti-cancer agents are three or more anti-cancer agents.

31. The method of claim 28, wherein the one or more anti-cancer agents are four or more anti-cancer agents.

32. The method of any one of the preceding claims, wherein the anti-cancer therapy is one or more anti-cancer therapies.

33. The method of claim 32, wherein the one or more anti-cancer therapies are two or more anti-cancer therapies.

34. The method of claim 32, wherein the one or more anti-cancer therapies are three or more anti-cancer therapies.

35. The method of claim 32, wherein the one or more anti-cancer therapies are four or more anti-cancer therapies.

36. The method of any one of the preceding claims, wherein the anti-cancer therapy is selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, and combinations thereof.

37. The method of any one of claims 32-35, wherein the one or more anti-cancer therapies are selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, and combinations thereof.

38. The method of any one of the preceding claims, wherein the anti-cancer agent is selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof.

39. The method of any one of claims 28-37, wherein the one or more anti-cancer agents are selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof.

40. The method of claim 38 or 39, wherein the chemotherapeutic or growth inhibitory agent is selected from the group consisting of an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof.

41. The method of claim 38 or 39, wherein the targeted therapeutic agent is selected from the group consisting of a B-raf inhibitor, a MEK inhibitor, a K-ras inhibitor, a c-Met inhibitor, an Alk inhibitor, a phosphatidylinositol 3-kinase inhibitor, an Akt inhibitor, an mTOR inhibitor, a dual phosphatidylinositol 3-kinase/mTOR inhibitor, and combinations thereof.

42. The method of claim 38 or 39, wherein the T cell expressing a chimeric antigen receptor comprises a dominant-negative TGF beta receptor.

43. The method of claim 38 or 39, wherein the antibody or antigen-binding fragment thereof is selected from the group consisting of alemtuzumab, bevacizumab, cetuximab, panitumumab, rituximab, pertuzumab, trastuzumab, tositumomab, apolizumab, aselizumab, atlizumab, bapineuzumab, cedelizumab, certolizumab pegol, cidfusituzumab, cidtuzumab, clivatuzumab, daclizumab, eculizumab, efalizumab, epratuzumab, erlizumab, felvizumab, fontolizumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pertuzumab, pexelizumab, ralivizumab, ranibizumab, reslivizumab, reslizumab, resyvizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, anti-IL-12, and anti-IL-17.

44. The method of claim 38 or 39, wherein the antibody or antigen-binding fragment thereof specifically binds to a target selected from the group consisting of CD52, VEGF-A, EGFR, CD20, HER2, HLA-DRB, CD62L, IL-6R, amyloid beta, CD44, CanAg, CD4, TNF alpha, IL-2, CD25, complement C5, CD11a, CD22, CD18, respiratory syncytial virus F, interferon gamma, CD33, CEACAM5, IL-5, integrin alpha 4, IgE, IL-4, IL-5, CD154, FAP, CD2, MUC-1, AFP, integrin αIIbβ3, ClfA, IL6R, CD40L, EpCAM, Shiga-like toxin II, IL-12, IL-23, IL-17, and CD3.

45. The method of claim 38 or 39, wherein the antibody-drug conjugate comprises a drug selected from the group consisting of mertansine, monomethyl auristatin E, a calicheamicin, an esperamicin, and a radioisotope chelator.

46. The method of claim 38 or 39, wherein the angiogenesis inhibitor is selected from the group consisting of a VEGF antagonist and an angiopoietin 2 antagonist.

47. The method of claim 38 or 39, wherein the antineoplastic agent is selected from the group consisting of an agent targeting CSF-1R, an interferon, GM-CSF, IL-2, IL-12, and an antibody targeting CD20.

48. The method of claim 38 or 39, wherein the cancer vaccine is selected from the group consisting of a peptide cancer vaccine, a personalized peptide vaccine, a multivalent long peptide vaccine, a multi-peptide vaccine, a peptide cocktail vaccine, a hybrid peptide vaccine, and a peptide-pulsed dendritic cell vaccine.

49. The method of any one of claims 1-37, wherein the anti-cancer agent is selected from the group consisting of a TLR agonist, tumor necrosis factor alpha, IL-1, HMGB1, an IL-10 antagonist, an IL-4 antagonist, an IL-13 antagonist, a treatment targeting CX3CL1, a treatment targeting CXCL9, a treatment targeting CXCL10, a treatment targeting CCL5, an LFA-1 agonist, an ICAM1 agonist, and a Selectin agonist.

50. The method of any one of claims 28-37, wherein the one or more anti-cancer agents are selected from the group consisting of a TLR agonist, tumor necrosis factor alpha, IL-1, HMGB1, an IL-10 antagonist, an IL-4 antagonist, an IL-13 antagonist, a treatment targeting CX3CL1, a treatment targeting CXCL9, a treatment targeting CXCL10, a treatment targeting CCL5, an LFA-1 agonist, an ICAM1 agonist, a Selectin agonist, and combinations thereof.

51. The method of any one of claims 1-50, wherein the agent that decreases or inhibits TIGIT expression and/or activity is administered continuously.

52. The method of any one of claims 1-50, wherein the agent that decreases or inhibits TIGIT expression and/or activity is administered intermittently.

53. The method of any one of claims 1-52, wherein the anti-cancer agent or anti-cancer therapy is administered continuously.

54. The method of any one of claims 1-52, wherein the anti-cancer agent or anti-cancer therapy is administered intermittently.

55. The method of any one of the preceding claims, wherein the agent that decreases or inhibits TIGIT expression and/or activity is administered before the anti-cancer agent or anti-cancer therapy.

56. The method of any one of the preceding claims, wherein the agent that decreases or inhibits TIGIT expression and/or activity is administered simultaneous with the anti-cancer agent or anti-cancer therapy.

57. The method of any one of the preceding claims, wherein the agent that decreases or inhibits TIGIT expression and/or activity is administered after the anti-cancer agent or anti-cancer therapy.

58. The method of any one of the preceding claims, wherein the cancer is selected from the group consisting of non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycosis fungoides, merkel cell cancer, and other hematologic malignancies.

59. The method of claim 58, wherein the cancer has elevated levels of T cell infiltration.

60. The method of any one of the preceding claims, wherein activated CD4 and/or CD8 T cells in the individual are characterized by γ-IFN⁺ producing CD4 and/or CD8 T cells and/or enhanced cytolytic activity relative to prior to the administration of the combination.

61. The method of claim 60, wherein the CD4 and/or CD8 T cells exhibit increased release of cytokines selected from the group consisting of IFN-γ, TNF-α and interleukins.

62. The method of claim 60 or 61, wherein the CD4 and/or CD8 T cells are effector memory T cells.

63. The method of claim 62, wherein the CD4 and/or CD8 effector memory T cells are characterized by having the expression of CD44^{high} CD62L^{low}.

64. A kit comprising an anti-cancer agent and a package insert comprising instructions for using the anti-cancer agent in combination with an agent that decreases or inhibits TIGIT expression and/or activity to treat or delay progression of cancer in an individual.

65. A kit comprising an anti-cancer agent and an agent that decreases or inhibits TIGIT expression and/or activity, and a package insert comprising instructions for using the anti-cancer agent and the agent that decreases or inhibits TIGIT expression and/or activity to treat or delay progression of cancer in an individual.

66. A kit comprising an agent that decreases or inhibits TIGIT expression and/or activity and a package insert comprising instructions for using the agent that decreases or inhibits TIGIT expression and/or activity in combination with an anti-cancer agent or an anti-cancer therapy to treat or delay progression of cancer in an individual.

67. A kit comprising an anti-cancer agent and a package insert comprising instructions for using the anti-cancer agent in combination with an agent that decreases or inhibits TIGIT expression and/or activity to reduce or inhibit cancer relapse or cancer progression in an individual having cancer.

68. A kit comprising an anti-cancer agent and an agent that decreases or inhibits TIGIT expression and/or activity, and a package insert comprising instructions for using the anti-cancer agent and the agent that decreases or inhibits TIGIT expression and/or activity to reduce or inhibit cancer relapse or cancer progression in an individual having cancer.

69. A kit comprising an agent that decreases or inhibits TIGIT expression and/or activity and a package insert comprising instructions for using the agent that decreases or inhibits TIGIT expression and/or activity in combination with an anti-cancer agent or an anti-cancer therapy to reduce or inhibit cancer relapse or cancer progression in an individual having cancer.

70. A kit comprising an anti-cancer agent and a package insert comprising instructions for using the anti-cancer agent in combination with an agent that decreases or inhibits TIGIT expression and/or activity to treat or delay progression of tumor immunity in an individual having cancer.

71. A kit comprising an anti-cancer agent and an agent that decreases or inhibits TIGIT expression and/or activity, and a package insert comprising instructions for using the anti-cancer agent and the agent that decreases or inhibits TIGIT expression and/or activity to treat or delay progression of tumor immunity in an individual having cancer.

72. A kit comprising an agent that decreases or inhibits TIGIT expression and/or activity and a package insert comprising instructions for using the agent that decreases or inhibits TIGIT expression and/or activity in combination with an anti-cancer agent or an anti-cancer therapy to treat or delay progression of tumor immunity in an individual having cancer.

73. A kit comprising an anti-cancer agent and a package insert comprising instructions for using the anti-cancer agent in combination with an agent that decreases or inhibits TIGIT expression and/or activity to increase, enhance, or stimulate an immune response or function in an individual having cancer.

74. A kit comprising an anti-cancer agent and an agent that decreases or inhibits TIGIT expression and/or activity, and a package insert comprising instructions for using the anti-cancer agent and the agent that decreases or inhibits TIGIT expression and/or activity to increase, enhance, or stimulate an immune response or function in an individual having cancer.

75. A kit comprising an agent that decreases or inhibits TIGIT expression and/or activity and a package insert comprising instructions for using the agent that decreases or inhibits TIGIT expression and/or activity in combination with an anti-cancer agent or an anti-cancer therapy to increase, enhance, or stimulate an immune response or function in an individual having cancer.

76. The kit of any one of claims 64-75, wherein the anti-cancer agent is one or more anti-cancer agents.

77. The kit of claim 76, wherein the one or more anti-cancer agents are two or more anti-cancer agents.

78. The kit of claim 76, wherein the one or more anti-cancer agents are three or more anti-cancer agents.

79. The kit of claim 76, wherein the one or more anti-cancer agents are four or more anti-cancer agents.

80. The kit of any one of claims 66, 69, 72, or 75, wherein the anti-cancer therapy is one or more anti-cancer therapies.

81. The kit of claim 80, wherein the one or more anti-cancer therapies are two or more anti-cancer therapies.

82. The kit of claim 80, wherein the one or more anti-cancer therapies are three or more anti-cancer therapies.

83. The kit of claim 80, wherein the one or more anti-cancer therapies are four or more anti-cancer therapies.

84. The kit of claim 66, 69, 72, or 75, wherein the anti-cancer therapy is selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, and combinations thereof.

85. The kit of claim 66, 69, 72, 75, or 80-83 wherein the one or more anti-cancer therapies are selected from the group consisting of radiation therapy, surgery, chemotherapy, gene therapy, DNA therapy, viral therapy, RNA therapy, immunotherapy, bone marrow transplantation, nanotherapy, monoclonal antibody therapy, adjuvant therapy, neoadjuvant therapy, and combinations thereof.

86. The kit of any one of claims 64-85, wherein the agent that decreases or inhibits TIGIT expression and/or activity is selected from the group consisting of an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, and an agent that inhibits and/or blocks the intracellular signaling mediated by TIGIT binding to PVRL3.

87. The kit of claim 86, wherein the antagonist of TIGIT expression and/or activity is an anti-TIGIT antibody or antigen-binding fragment thereof.

88. The kit of claim 87, wherein the anti-TIGIT antibody or antigen-binding fragment thereof comprises at least one HVR comprising an amino acid sequence selected from the amino acid sequences (1) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (2) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

89. The kit of claim 87 or 88, wherein the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14).

90. The kit of any one of claims 87-89, wherein the anti-TIGIT antibody or antigen-binding fragment thereof, wherein the antibody heavy chain comprises the amino acid sequence set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO:16).

91. The kit of any one of claims 87-89, wherein the anti-TIGIT antibody or antigen-binding fragment thereof, wherein the antibody light chain comprises the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14), and the antibody heavy chain comprises the amino acid sequence set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO: 16).

92. The kit of claim 87, wherein the anti-TIGIT antibody or antigen-binding fragment thereof comprises at least one HVR that is at least 90% identical to an HVR set forth in any one of (1) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (2) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

93. The kit of claim 92, wherein the anti-TIGIT antibody or fragment thereof comprises the light chain comprising amino acid sequences at least 90% identical to the amino acid sequences set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTK LEIKR (SEQ ID NO:13) or DVVLTQTPLSLSVSFGDQVSISCRSSQSLVNSYGNTFLSWYLHKPGQSPQLLIFGISNRFS GVPDRFSGSGSGTDFTLKISTIKPEDLGMYYCLQGTHQPPTFGPGTKLEVK (SEQ ID NO:14); and/or the heavy chain comprising amino acid sequences at least 90% identical to the amino acid sequences set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVT VSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVT VSS (SEQ ID NO:16).

94. The kit of any one of claims 64-93, wherein the anti-cancer agent is selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof.

95. The kit of any one of claims 76-93, wherein the one or more anti-cancer agents are selected from the group consisting of a chemotherapeutic or growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, and combinations thereof.

96. The kit of claim 94 or 95, wherein the chemotherapeutic or growth inhibitory agent is selected from the group consisting of an alkylating agent, an anthracycline, an anti-hormonal agent, an aromatase inhibitor, an anti-androgen, a protein kinase inhibitor, a lipid kinase inhibitor, an antisense oligonucleotide, a ribozyme, an antimetabolite, a topoisomerase inhibitor, a cytotoxic agent or antitumor antibiotic, a proteasome inhibitor, an anti-microtubule agent, an EGFR antagonist, a retinoid, a tyrosine kinase inhibitor, a histone deacetylase inhibitor, and combinations thereof.

97. The kit of claim 94 or 95, wherein the targeted therapeutic agent is selected from the group consisting of a B-raf inhibitor, a MEK inhibitor, a K-ras inhibitor, a c-Met inhibitor, an Alk inhibitor, a phosphatidylinositol 3-kinase inhibitor, an Akt inhibitor, an mTOR inhibitor, a dual phosphatidylinositol 3-kinase/mTOR inhibitor, and combinations thereof.

98. The kit of claim 94 or 95, wherein the T cell expressing a chimeric antigen receptor comprises a dominant-negative TGF beta receptor.

99. The kit of claim 94 or 95, wherein the antibody or antigen-binding fragment thereof is selected from the group consisting of alemtuzumab, bevacizumab, cetuximab, panitumumab, rituximab, pertuzumab, trastuzumab, tositumomab, apolizumab, aselizumab, atlizumab, bapineuzumab, cedelizumab, certolizumab pegol, cidfusituzumab, cidtuzumab, clivatuzumab, daclizumab, eculizumab, efalizumab, epratuzumab, erlizumab, felvizumab, fontolizumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pertuzumab, pexelizumab, ralivizumab, ranibizumab, reslivizumab, reslizumab, resyvizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, anti-IL-12, and anti-IL-17.

100. The kit of claim 94 or 95, wherein the antibody or antigen-binding fragment thereof specifically binds to a target selected from the group consisting of CD52, VEGF-A, EGFR, CD20, HER2, HLA-DRB, CD62L, IL-6R, amyloid beta, CD44, CanAg, CD4, TNF alpha, IL-2, CD25, complement C5, CD11a, CD22, CD18, respiratory syncytial virus F, interferon gamma, CD33, CEACAM5, IL-5, integrin alpha 4, IgE, IL-4, IL-5, CD154, FAP, CD2, MUC-1, AFP, integrin αIIbβ3, ClfA, IL6R, CD40L, EpCAM, Shiga-like toxin II, IL-12, IL-23, IL-17, and CD3.

101. The kit of claim 94 or 95, wherein the antibody-drug conjugate comprises a drug selected from the group consisting of mertansine, monomethyl auristatin E, calicheamicin, esperamicin, and a radioisotope chelator.

102. The kit of claim 94 or 95, wherein the angiogenesis inhibitor is selected from the group consisting of a VEGF antagonist and an angiopoietin 2 antagonist.

103. The kit of claim 94 or 95, wherein the antineoplastic agent is selected from the group consisting of an agent targeting CSF-1R, an interferon, GM-CSF, IL-2, IL-12, and an antibody targeting CD20.

104. The kit of claim 94 or 95, wherein the cancer vaccine is selected from the group consisting of a peptide cancer vaccine, a personalized peptide vaccine, a multivalent long peptide vaccine, a multi-peptide vaccine, a peptide cocktail vaccine, a hybrid peptide vaccine, and a peptide-pulsed dendritic cell vaccine.

105. The kit of any one of claims 64-93, wherein the anti-cancer agent is selected from the group consisting of a TLR agonist, tumor necrosis factor alpha, IL-1, HMGB1, an IL-10 antagonist, an IL-4 antagonist, an IL-13 antagonist, a treatment targeting CX3CL1, a treatment targeting CXCL9, a treatment targeting CXCL10, a treatment targeting CCL5, an LFA-1 agonist, an ICAM1 agonist, and a Selectin agonist.

106. The kit of any one of claims 76-93, wherein the one or more anti-cancer agents are selected from the group consisting of a TLR agonist, tumor necrosis factor alpha, IL-1, HMGB1, an IL-10 antagonist, an IL-4 antagonist, an IL-13 antagonist, a treatment targeting CX3CL1, a treatment targeting CXCL9, a treatment targeting CXCL10, a treatment targeting CCL5, an LFA-1 agonist, an ICAM1 agonist, a Selectin agonist, and combinations thereof.


SEQUENCE LISTING (26 sequences)

SEQ ID NO: 1 - protein, 17 aa, synthetic construct
KSSQSLYYSGVKENLLA

SEQ ID NO: 2 - protein, 6 aa, synthetic construct
ASIRFT

SEQ ID NO: 3 - protein, 9 aa, synthetic construct
QQGINNPLT

SEQ ID NO: 4 - protein, 10 aa, synthetic construct
GFTFSSFTMH

SEQ ID NO: 5 - protein, 17 aa, synthetic construct
FIRSGSGIVFYADAVRG

SEQ ID NO: 6 - protein, 10 aa, synthetic construct
RPLGHNTFDS

SEQ ID NO: 7 - protein, 16 aa, synthetic construct
RSSQSLVNSYGNTFLS

SEQ ID NO: 8 - protein, 7 aa, synthetic construct
GISNRFS

SEQ ID NO: 9 - protein, 9 aa, synthetic construct
LQGTHQPPT

SEQ ID NO: 10 - protein, 10 aa, synthetic construct
GYSFTGHLMN

SEQ ID NO: 11 - protein, 17 aa, synthetic construct
LIIPYNGGTSYNQKFKG

SEQ ID NO: 12 - protein, 10 aa, synthetic construct
GLRGFYAMDY

SEQ ID NO: 13 - protein, 114 aa, synthetic construct
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKR

SEQ ID NO: 14 - protein, 112 aa, synthetic construct
DVVLTQTPLSLSVSFGDQVSISCRSSQSLVNSYGNTFLSWYLHKPGQSPQLLIFGISNRFSGVPDRFSGSGSGTDFTLKISTIKPEDLGMYYCLQGTHQPPTFGPGTKLEVK

SEQ ID NO: 15 - protein, 119 aa, synthetic construct
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSS

SEQ ID NO: 16 - protein, 119 aa, synthetic construct
EVQLQQSGPELVKPGTSMKISCKASGYSFTGHLMNWVKQSHGKNLEWIGLIIPYNGGTSYNQKFKGKATLTVDKSSSTAYMELLSLTSDDSAVYFCSRGLRGFYAMDYWGQGTSVTVSS

SEQ ID NO: 17 - DNA, 336 bp, synthetic construct
  source 1..336: mol_type = other DNA
gatgttgtgttgactcaaactccactctccctgtctgtcagctttggagatcaagtttctatctcttgcaggtctagtcagagtcttgtaaacagttatgggaacacctttttgtcttggtacctgcacaagcctggccagtctccacagctcctcatctttgggatttccaacagattttctggggtgccagacaggttcagtggcagtggttcagggacagatttcacactcaagatcagcacaataaagcctgaggacttgggaatgtattactgcttacaaggtacgcatcagcctcccacgttcggtcctgggaccaagctggaggtgaaa

SEQ ID NO: 18 - DNA, 357 bp, synthetic construct
  source 1..357: mol_type = other DNA
gaggtccagctgcaacagtctggacctgagctggtgaagcctggaacttcaatgaagatatcctgcaaggcttctggttactcattcactggccatcttatgaactgggtgaagcagagccatggaaagaaccttgagtggattggacttattattccttacaatggtggtacaagctataaccagaagttcaagggcaaggccacattgactgtagacaagtcatccagcacagcctacatggagctcctcagtctgacttctgatgactctgcagtctatttctgttcaagaggccttaggggcttctatgctatggactactggggtcaaggaacctcagtcaccgtctcctca

SEQ ID NO: 19 - protein, 25 aa, synthetic construct [sequence omitted]

SEQ ID NO: 20 - protein, 13 aa, synthetic construct
WVRQAPGKGLEWV

SEQ ID NO: 21 - protein, 32 aa, synthetic construct
RFTISADTSKNTAYLQMNSLRAEDTAVYYCAR

SEQ ID NO: 22 - protein, 11 aa, synthetic construct
WGQGTLVTVSA

SEQ ID NO: 23 - protein, 23 aa, synthetic construct
DIQMTQSPSSLSASVGDRVTITC

SEQ ID NO: 24 - protein, 15 aa, synthetic construct
WYQQKPGKAPKLLIY

SEQ ID NO: 25 - protein, 32 aa, synthetic construct
GVPSRFSGSGSGTDFTLTISSLQPEDFATYYC

SEQ ID NO: 26 - protein, 11 aa, synthetic construct
FGQGTKVEIKR